(19) United States
(12) Patent Application Publication
(10) Pub. No.: US 2018/0078184 A1
(43) Pub. Date: Mar. 22, 2018

(54) DUAL-TASK PERFORMING ABILITY EVALUATION METHOD AND DUAL-TASK PERFORMING ABILITY EVALUATION SYSTEM

(71) Applicant: OSAKA UNIVERSITY, Osaka (JP)

(72) Inventors: Yasushi YAGI, Suita-shi (JP); Ikuhisa MITSUGAMI, Suita-shi (JP); Hirotake YAMAZOE, Suita-shi (JP); Mitsuru NAKAZAWA, Suita-shi (JP); Masataka NIWA, Suita-shi (JP)

(73) Assignee: OSAKA UNIVERSITY, Osaka (JP)

(21) Appl. No.: 15/558,607

(22) PCT Filed: Mar. 16, 2016
(86) PCT No.: PCT/JP2016/058353

§ 371 Date: Sep. 15, 2017

(30) Foreign Application Priority Data
Mar. 16, 2015 (JP) 2015-052205

(51) Int. Cl. A61B 5/11; G16H 50/20

(52) U.S. Cl. CPC A61B 5/1124; G16H 50/20; A61B 10/00; G01N 2800/50; A61H 2201/503; G01N 2800/2814

(57) ABSTRACT

A dual-task performing ability evaluation method includes a dual task step (S201), an analysis step (S401), and an evaluation step (S601). In the dual task step (S201), a subject performs a dual task including a movement task that requires the subject to perform specific movement and an intelligence task that requires the subject to answer a specific problem. Further, at least one of motion and an answer of the subject performing the dual task is detected. In the analysis step (S401), the at least one of the motion and the answer that has been detected is analyzed. In the evaluation step (S601), dual-task performing ability of the subject is evaluated based on a result of the analysis.

### TECHNICAL FIELD

The present invention relates to a dual-task performing ability evaluation method and a dual-task performing ability evaluation system.

### BACKGROUND ART

Dementia is one of mental disorders. When dementia develops, higher-order brain functions (cognitive functions) such as memory, understanding, and judgement persistently decline. However, progress of dementia can be prevented or suppressed by detecting dementia at an early stage and initiating rehabilitation early. Therefore, it is important to detect dementia at an early stage (mild stage).

Dementia is typically diagnosed by evaluating cognitive functions. Cognitive functions are typically evaluated through medical interviews and intelligence tests. However, medical interviews and intelligence tests take much time for evaluation and therefore are not convenient.

By contrast, nerve image processing technologies such as magnetic resonance imaging and computer tomographic imaging are sometimes used for diagnosis of dementia (see Patent Literature 1, for example). However, large-scale apparatuses are necessary in the case of using the nerve image processing technologies.

By the way, dual tasks have attracted attention in recent years as a rehabilitation menu for dementia. A dual task refers to using one's intellect while moving their body. For example, it has been found that doing serial subtraction while walking is effective as rehabilitation of dementia. Serial subtraction refers to successively subtracting 1 from a preceding value by starting from 100, for example.

### CITATION LIST

### Patent Literature

[Patent Literature 1] Japanese Patent Application Laid-Open Publication No. 2014-197018

### SUMMARY OF INVENTION

### Technical Problem

The present inventors have actually observed a number of elderly people working on a dual task. Also, the present inventors have further advanced research and observed a number of healthy people working on a dual task. From the above experience, the present inventors have developed a novel method for evaluating ability of a subject to perform a dual task and completed the present invention.

An object of the present invention is to provide a dual-task performing ability evaluation method that permits evaluation of ability of a subject to perform a dual task, and a dual-task performing ability evaluation system.

### Solution to Problem

A dual-task performing ability evaluation method according to the present invention includes a dual task step, an analysis step, and an evaluation step. In the dual task step, a subject performs a dual task including a movement task that requires the subject to perform specific movement and an intelligence task that requires the subject to answer a specific problem. Also, at least one of motion and an answer of the subject performing the dual task is detected. In the analysis step, analysis of the at least one of the motion and the answer that has been detected is performed. In the evaluation step, dual-task performing ability of the subject is evaluated based on a result of the analysis.

According to an embodiment, in the analysis step, an answer score of the subject is calculated based on the answer detected in the dual task step, and in the evaluation step, the dual-task performing ability of the subject is evaluated based on the answer score.

According to an embodiment, the dual-task performing ability evaluation method further includes a movement task step. In the movement task step, the subject performs only the movement task. Motion of the subject performing only the movement task is detected. In the analysis step, determination is made on whether or not a state of movement of the subject is consistent between the dual task step and the movement task step with respect to a specific evaluation item, based on the motion detected in the movement task step and the motion detected in the dual task step. In the evaluation step, the dual-task performing ability of the subject is evaluated based on a result of the determination.

According to an embodiment, the dual-task performing ability evaluation method further includes a movement task step. In the movement task step, the subject performs only the movement task. Motion of the subject performing only the movement task is detected. The dual task step includes a first dual task step and a second dual task step. In the first dual task step, the subject performs a first dual task including the movement task and a first intelligence task that requires the subject to answer a specific first problem. Motion of the subject performing the first dual task is detected. In the second dual task step, the subject performs a second dual task including the movement task and a second intelligence task that requires the subject to answer a specific second problem. Motion of the subject performing the second dual task is detected. A difficulty level of the second intelligence task is higher than that of the first intelligence task. In the analysis step, determination is made on whether or not evaluation results of at least two of plural evaluation items each indicating a state of movement of the subject become lower in order of the movement task step, the first dual task step, and the second dual task step, based on the motion detected in the movement task step, the motion detected in the first dual task step, and the motion detected in the second dual task step. In the evaluation step, the dual-task performing ability of the subject is evaluated based on a result of the determination.

According to an embodiment, in the analysis step, determination is made on whether or not the subject stopped the specific movement based on the motion detected in the dual task step, and in the evaluation step, the dual-task performing ability of the subject is evaluated based on a result of the determination.

According to an embodiment, in the dual task step, the subject performs the dual task for a specific period of time. In the analysis step, determination is made on whether or not the subject stopped the specific movement and finished answering the specific problem before the specific period of time elapsed, based on the motion and the answer that have been detected in the dual task step. In the evaluation step, the dual-task performing ability of the subject is evaluated based on a result of the determination.

According to an embodiment, in the analysis step, variation of intervals between answers of the subject is determined based on answers detected in the dual task step, and in the evaluation step, the dual-task performing ability of the subject is evaluated based on a result of the determination.

According to an embodiment, an MMSE score or a Hasegawa's Dementia Scale is determined as the dual-task performing ability.

According to an embodiment, in the analysis step, a characteristic amount of the motion detected in the dual task step or a characteristic amount of the answer detected in the dual task step is measured. The measured characteristic amount is compared with a standard value for the characteristic amount. In the evaluation step, the dual-task performing ability of the subject is evaluated based on a result of the comparison.

According to an embodiment, in the analysis step, the measured characteristic amount is compared with a standard value for an actual age of the subject.

A first dual-task performing ability evaluation system of the present invention includes an answer detecting section and an evaluation data generating section. The answer detecting section detects an answer of a subject performing a dual task including a movement task that requires the subject to perform specific movement and an intelligence task that requires the subject to answer a specific problem. The evaluation data generating section generates evaluation data indicating dual-task performing ability of the subject based on a result of analysis of the answer detected by the answer detecting section.

According to an embodiment, the first dual-task performing ability evaluation system further includes an analyzing section that analyzes the answer detected by the answer detecting section.

According to an embodiment, the analyzing section compares a characteristic amount of the answer with a standard value for the characteristic amount.

According to an embodiment, the first dual-task performing ability evaluation system further includes a motion detecting section that detects motion of the subject performing the dual task. The evaluation data generating section generates the evaluation data based on at least one of the result of analysis of the answer detected by the answer detecting section and a result of analysis of the motion detected by the motion detecting section.

According to an embodiment, the first dual-task performing ability evaluation system further includes an analyzing section that analyzes at least one of the answer detected by the answer detecting section and the motion detected by the motion detecting section.

According to an embodiment, the analyzing section compares a characteristic amount of the answer or a characteristic amount of the motion with a standard value for the characteristic amount.

A second dual-task performing ability evaluation system of the present invention includes a motion detecting section and an evaluation data generating section. The motion detecting section detects motion of a subject performing a dual task including a movement task that requires the subject to perform specific movement and an intelligence task that requires the subject to answer a specific problem. The evaluation data generating section generates evaluation data indicating dual-task performing ability of the subject based on a result of analysis of the motion detected by the motion detecting section.

According to an embodiment, the second dual-task performing ability evaluation system further includes an analyzing section that analyzes the motion detected by the motion detecting section.

According to an embodiment, the analyzing section compares a characteristic amount of the motion with a standard value for the characteristic amount.

According to an embodiment, the motion detecting section further detects motion of the subject performing only a movement task that requires the subject to perform the specific movement.

According to an embodiment, the dual task includes plural kinds of dual tasks. The motion detecting section detects motion of the subject performing each of the plural kinds of dual tasks.

### Advantageous Effects of Invention

According to the present invention, ability of a subject to perform a dual task can be evaluated.

### BRIEF DESCRIPTION OF DRAWINGS

FIG. 1 is a diagram illustrating a flow of a dual-task performing ability evaluation method according to a first embodiment of the present invention.

FIG. 2 is a block diagram illustrating configuration of a dual-task performing ability evaluation system according to the first embodiment and a sixth embodiment of the present invention.

FIG. 3 is a diagram illustrating a flow of a dual-task performing ability evaluation method according to a second embodiment of the present invention.

FIG. 4 is a block diagram illustrating configuration of a dual-task performing ability evaluation system according to the second through fourth embodiments of the present invention.

FIG. 5 is a diagram illustrating a human body skeleton model according to an embodiment of the present invention.

FIG. 6 is a diagram illustrating human body skeleton models displayed on a display section of an information processing device according to the second and fourth embodiments of the present invention.

FIG. 7 is a diagram illustrating a flow of a dual-task performing ability evaluation method according to a third embodiment of the present invention.

FIG. 8 is a diagram illustrating human body skeleton models displayed on a display section of an information processing device according to the third embodiment of the present invention.

FIG. 9 is a diagram illustrating a flow of a dual-task performing ability evaluation method according to the fourth embodiment of the present invention.

FIG. 10 is a diagram illustrating a flow of a dual-task performing ability evaluation method according to a fifth embodiment of the present invention.

FIG. 11 is a block diagram illustrating configuration of a dual-task performing ability evaluation system according to the fifth embodiment of the present invention.

FIG. 12 is a diagram illustrating an image displayed on a display section of an information processing device according to the fifth embodiment of the present invention.

FIG. 13 is a diagram illustrating a flow of a dual-task performing ability evaluation method according to the sixth embodiment of the present invention.

FIG. 14 is a diagram illustrating configuration of a dual-task performing ability evaluation system according to a seventh embodiment of the present invention.

FIG. 15 is a diagram illustrating an example of a task presented to a subject by the dual-task performing ability evaluation system according to the seventh embodiment of the present invention.

FIG. 16 is a diagram illustrating another example of a task presented to a subject by the dual-task performing ability evaluation system according to the seventh embodiment of the present invention.

FIG. 17 is a diagram illustrating a flow of a dual-task performing ability evaluation method according to an eighth embodiment of the present invention.

FIG. 18 is a diagram illustrating configuration of a dual-task performing ability evaluation system according to the eighth embodiment of the present invention.

FIG. 19 is a diagram illustrating an example of standard value data according to the eighth embodiment of the present invention.

FIG. 20 is a diagram illustrating measurement results according to a first example of the present invention.

FIGS. 21A to 21C are diagrams each illustrating measurement results according to a fifth example of the present invention.

FIGS. 22A to 22C are diagrams each illustrating measurement results according to the fifth example of the present invention.

FIGS. 23A to 23C are diagrams each illustrating measurement results according to the fifth example of the present invention.

FIGS. 24A to 24C are diagrams each illustrating measurement results according to the fifth example of the present invention.

FIGS. 25A and 25B are diagrams each illustrating measurement results according to the fifth example of the present invention.

FIGS. 26A and 26B are diagrams each illustrating measurement results according to the fifth example of the present invention.

FIG. 27 is a diagram illustrating measurement results according to the fifth example of the present invention.

FIG. 28 is a diagram illustrating measurement results according to a sixth example of the present invention.

FIG. 29 is a diagram illustrating data of subjects according to a seventh example of the present invention.

FIG. 30 is a diagram illustrating measurement results according to the seventh example of the present invention.

FIG. 31 is a diagram illustrating measurement results according to the seventh example of the present invention.

FIG. 32 is a diagram illustrating data of subjects according to an eighth example of the present invention.

FIG. 33 is a diagram illustrating measurement results according to the eighth example of the present invention.

FIG. 34 is a diagram illustrating measurement results according to the eighth example of the present invention.

FIG. 35 is a diagram illustrating measurement results according to the eighth example of the present invention.

FIG. 36 is a diagram illustrating measurement results according to the eighth example of the present invention.

### DESCRIPTION OF EMBODIMENTS

The following explains embodiments of the present invention with reference to the drawings. Elements in the drawings that are the same or equivalent are marked by the same reference signs. Furthermore, explanation of such elements is not repeated. The drawings are schematic illustrations that emphasize elements of configuration in order to facilitate understanding thereof.

### First Embodiment

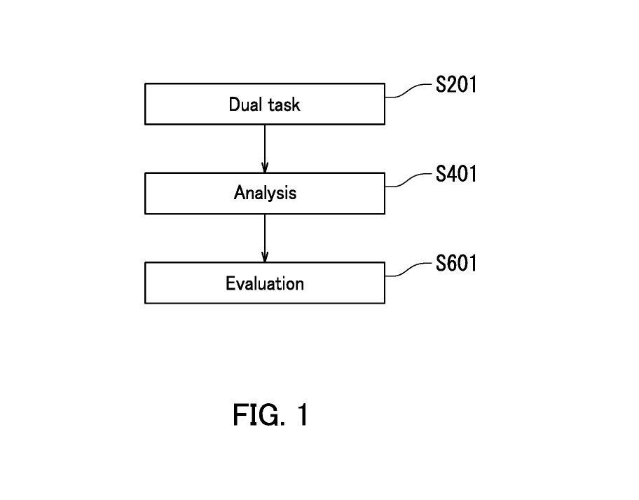

Initially, a dual-task performing ability evaluation method according to a first embodiment will be explained with reference to FIG. 1. FIG. 1 is a diagram illustrating a flow of the dual-task performing ability evaluation method according to the first embodiment. As illustrated in FIG. 1, the dual-task performing ability evaluation method according to the first embodiment includes a dual task step S201, an analysis step S401, and an evaluation step S601.

In the dual task step S201, a subject performs a dual task for a specific period of time. The dual task includes a movement task and an intelligence task. The movement task requires the subject to perform specific movement. The intelligence task requires the subject to answer a specific problem. In the dual task step S201, the subject simultaneously performs the movement task and the intelligence task. Further, in the dual task step S201, an answer of the subject performing the dual task is detected.

The specific movement that the movement task requires the subject to perform is for example “straight walking”. Alternatively, the specific movement may be “walking in place”, “skipping”, or “running”. A problem that the intelligence task requires the subject to answer is for example a calculation problem. Alternatively, a problem that the intelligence task requires the subject to answer may be a problem to be answered with “language”.

The calculation problem is for example “serial subtraction”. Alternatively, the calculation problem may be “calculation using a one-digit number and a one-digit number”, “calculation using a one-digit number and a two-digit number”, or “calculation using a two-digit number and a two-digit number”. The problem to be answered with “language” may be “recitation of expressions (e.g., words) starting from a syllable (letter) randomly chosen from the Japanese syllabary” or a “word-chain game”. Alternatively, the problem to be answered with “language” may be “recitation of expressions (e.g., words) starting from a letter randomly chosen from alphabets”.

In the analysis step S401, an answer of the subject to the intelligence task is analyzed. Specifically, in the first embodiment, an answer score of the subject is calculated. The answer score may for example be the number of answers including wrong answers, the number of right answers (i.e., the number of correct answers), or a right answer ratio. The number of answers including wrong answers may be referred to below as a total number of answers. The right answer ratio indicates a ratio of right answers to the total number of answers. When a problem is answered with “language”, more points may be added for an answer including more letters.

In the evaluation step S601, dual-task performing ability of the subject is evaluated based on a result of the analysis obtained in the analysis step S401. Specifically, in the first embodiment, evaluation data that indicates the dual-task performing ability of the subject is generated based on the answer score obtained in the analysis step S401.

The dual-task performing ability evaluated in the evaluation step S601 indicates a degree of mental disorders of the subject, cognitive ability of the subject, or a health degree of the subject's brain. For example, the dual-task performing ability corresponds to a commonly used intelligence evaluation scale such as a Mini Mental State Examination (MMSE) score or a Hasegawa's Dementia Scale.

An intelligence evaluation scale can be calculated according to Formula (1) shown below based on the answer score. In Formula (1), “x” indicates the answer score. Also, f(x) is any function relating to the answer score.

Intelligence evaluation scale=f(x) (1)

For example, a linear polynomial represented by Formula (2) shown below may be employed as a formula for calculating the intelligence evaluation scale.

Intelligence evaluation scale=a×(x)+b (2)

The coefficients “a” and “b” in Formula (2) may change according to difficulty levels of the movement task and the intelligence task. Also, in the case of calculating an MMSE score as the intelligence evaluation scale, Formula (2) turns to Formula (3) shown below. That is, the smallest one of a value obtained according to the function “a×(x)+b” and a value “30” is taken to be the MMSE score.

MMSE score=min{a×(x)+b,30} (3)

For example, when straight walking for one minute (a movement task) and serial subtraction of successively subtracting 1 from a preceding value by starting from 100 (an intelligence task) are imposed as a dual task on the subject, the coefficient “a” in Formula (3) is “0.17”. Also, the coefficient “b” is “17.6”. Accordingly, Formula (3) turns to Formula (4) shown below. In this case, the function “0.17×x+17.6” corresponds to MMSE scores smaller than 27.

MMSE score=min{0.17×x+17.6,27} (4)

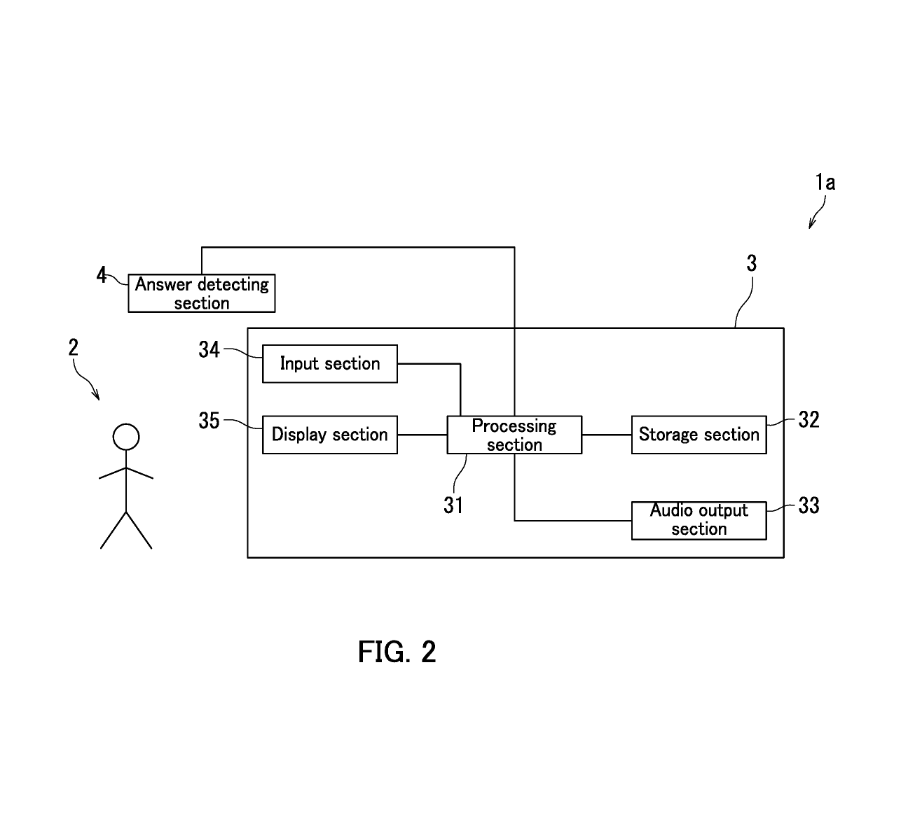

Next, a dual-task performing ability evaluation system 1a according to the first embodiment will be explained with reference to FIG. 2. FIG. 2 is a block diagram illustrating configuration of the dual-task performing ability evaluation system 1a. The dual-task performing ability evaluation method illustrated in FIG. 1 is performed using the dual-task performing ability evaluation system 1a. As illustrated in FIG. 2, the dual-task performing ability evaluation system 1a includes an information processing device 3 and an answer detecting section 4.

The information processing device 3 includes a processing section 31, a storage section 32, an audio output section 33, an input section 34, and a display section 35. The information processing device 3 may include for example a personal computer. Alternatively, the information processing device 3 may include a device that can process information such as a smartphone or a tablet terminal. The answer detecting section 4 may be incorporated in the information processing device 3.

The processing section 31 performs various processing such as numerical calculation, information processing, and device control by executing programs stored in the storage section 32. The processing section 31 may include for example a central processing unit (CPU) or a micro processing unit (MPU).

The storage section 32 stores therein programs, setting information, and the like. The storage section 32 may for example be constituted by a magnetic disk included in a hard disk drive (HDD), a random access memory (RAM) device, and a read only memory (ROM) device.

The processing section 31 can cause the audio output section 33 to output sound based on audio data stored in the storage section 32. The audio output section 33 may for example be a speaker.

The input section 34 is operated by a user to input various information to the processing section 31. The processing section 31 executes various processing based on information input through the input section 34. The input section 34 may include for example a keyboard and/or a touch panel.

The display section 35 can display various types of images under control of the processing section 31. The display section 35 may for example be a liquid crystal display or an organic EL display.

The answer detecting section 4 detects an answer of a subject 2. In the first embodiment, the answer detecting section 4 is a microphone. Accordingly, the answer detecting section 4 converts voice (an answer) uttered by the subject 2 performing an intelligence task to an electrical signal. The processing section 31 converts the electrical signal (analog signal) generated by the answer detecting section 4 (microphone) to a digital signal of a specific form to generate audio data. The storage section 32 stores therein the audio data generated by the processing section 31.

Next, a method for evaluating dual-task performing ability of the subject 2 by using the dual-task performing ability evaluation system 1a will be explained. Specifically, a method for determining (calculating) an intelligence evaluation scale of the subject 2 will be explained. In determining the intelligence evaluation scale of the subject 2, a user initially uses the information processing device 3 and the answer detecting section 4 to store (record) in the storage section 32 audio data corresponding to voice uttered by the subject 2 performing a dual task (e.g., straight walking and a calculation problem) for a specific period of time.

Next, the user operates the input section 34 to cause the audio output section 33 to output sound corresponding to the audio data stored in the storage section 32. In other words, the voice uttered by the subject 2 while performing the dual task is reproduced. The user calculates an answer score of the subject 2 based on the reproduced voice. In other words, an answer of the subject 2 is analyzed.

Next, the user operates the input section 34 to input data indicating the answer score (an analysis result). In the first embodiment, the storage section 32 stores therein Formula (1) shown above. The processing section 31 generates data indicating the intelligence evaluation scale of the subject 2 based on Formula (1) and the answer score. Then, the processing section 31 generates an image corresponding to the intelligence evaluation scale and causes the display section 35 to display the generated image. Thus, the processing section 31 generates data indicating the intelligence evaluation scale of the subject 2 as evaluation data that indicates dual-task performing ability of the subject 2. As such, the processing section 31 functions as an evaluation data generating section that generates the evaluation data. An image corresponding to the evaluation data will be referred to below as an evaluation result image.

As described above, according to the first embodiment, dual-task performing ability corresponding to an intelligence evaluation scale can be evaluated by having a subject perform a dual task. Therefore, a degree of dementia can be evaluated more easily in a shorter time as compared with a case where dementia is diagnosed through a medical interview such as an MMSE or an intelligence test.

Also, the system for evaluating the dual-task performing ability corresponding to an intelligence evaluation scale can be constituted by the information processing device 3 such as a personal computer and the answer detecting section 4 such as a microphone. Therefore, the system can be simplified as compared with a case where a mental disorder such as dementia is diagnosed by using nerve image processing technologies such as magnetic resonance imaging and computer tomographic imaging.

Also, the subject 2 can individually evaluate the dual-task performing ability corresponding to an intelligence evaluation scale by using the dual-task performing ability evaluation system 1a. Therefore, the subject 2 can keep a daily record of progress of a mental disorder such as dementia. Through the above, for example a worsening trend of the mental disorder such as dementia can be detected early to conduct appropriate rehabilitation and medical treatment. Consequently, progress of the mental disorder such as dementia can be suppressed or prevented.

In the first embodiment, the dual-task performing ability evaluation system 1a is constituted by the information processing device 3 such as a personal computer and the microphone (answer detecting section 4). However, elements of configuration of the dual-task performing ability evaluation system 1a are not limited to those described above. For example, devices such as an IC recorder and a smartphone may be used as an element that implements a function of recording audio data. Alternatively, devices that can process information such as a smartphone and a tablet terminal may for example be used as an element that implements a function of the evaluation data generating section.

Also, the evaluation result image is displayed on the display section 35 (data output section) included in the information processing device 3. However, the evaluation result image may be displayed for example on a display (data output section) that is connected as an external device to the information processing device 3. Also, the evaluation result image may for example be printed on a recording medium by a printer (data output section) that is connected as an external device to the information processing device 3.

Also, in the above-explained embodiment, the user calculates the answer score of the subject 2 by hearing the reproduced voice. However, the present invention is not limited to the above configuration. For example, when a total number of answers is counted as the answer score, the processing section 31 may generate data that indicates volume in time series and cause the display section 35 to display an image corresponding to the generated data. Through the above, the user can count the total number of answers based on the image (peak values) displayed on the display section 35. Alternatively, the processing section 31 may count the total number of answers by voice recognition processing. That is, the processing section 31 may function as an analyzing section to analyze an answer (voice) of the subject 2 and generate data indicating the total number of answers (data indicating an analysis result). In the configuration in which the processing section 31 functions as the analyzing section, the user need not input data indicating an analysis result.

Also, in the first embodiment, the intelligence task requires the subject to answer by utterance. However, the intelligence task may require the subject to answer by motion. For example, when walking in place is employed as the movement task, it is possible to employ an intelligence task of changing positions of putting down feet on the ground according to instructions of an examiner. Alternatively, a dual task may include an intelligence task of raising or lowering one or both arm(s) according to instructions of an examiner. Alternatively, a dual task may include an intelligence task of changing a viewing direction according to instructions of an examiner. Alternatively, a dual task may include an intelligence task that makes a subject hold switches in respective hands and press the switches according to instructions of an examiner. Alternatively, a dual task may include an intelligence task that makes a subject hold a switch in one hand and press the switch according to instructions of an examiner.

Also, in the first embodiment explained above, the dual-task performing ability evaluation system 1a includes the microphone as the answer detecting section 4. However, the dual-task performing ability evaluation system 1a is not limited to the above configuration.

For example, when the intelligence task requires the subject to answer by motion, the dual-task performing ability evaluation system 1a includes, as the answer detecting section 4, a motion detecting section that detects motion of the subject 2. Specifically, the motion detecting section may for example be an imaging device or a motion capture device. The imaging device is for example a digital video camera. The motion capture device captures (detects) movement of parts of the subject 2 by a motion capture technology.

In a configuration in which the answer detecting section 4 (motion detecting section) is an imaging device, the answer detecting section 4 generates image data of the subject 2 imaged by the imaging device. In the above configuration, the processing section 31 causes for example the display section 35 to display an image of the subject 2 imaged by the imaging device. Through the above, a user of the dual-task performing ability evaluation system 1a can check motion of the subject 2 and calculate an answer score of the subject 2.

In a configuration in which the answer detecting section 4 (motion detecting section) is a motion capture device, the answer detecting section 4 generates motion capture data reflecting motion of the subject 2. In the above configuration, the processing section 31 generates data indicating a human body skeleton model that moves along with the motion of the subject 2, based on the motion capture data. Data indicating a human body skeleton model may be referred to below as human body skeleton model data. The processing section 31 generates an image of the human body skeleton model based on the human body skeleton model data and causes for example the display section 35 to display the generated image. Through the above, a user of the dual-task performing ability evaluation system 1a can check the motion of the subject 2 (human body skeleton model data) and calculate an answer score of the subject 2.

In the configuration in which the dual-task performing ability evaluation system 1a includes a motion capture device, the processing section 31 may function as an analyzing section and calculate an answer score of the subject 2. That is, the processing section 31 may analyze the human body skeleton model data or the motion capture data and generate data indicating an answer score (data indicating an analysis result).

When the intelligence task is a task of changing a viewing direction according to instructions of an examiner, the dual-task performing ability evaluation system 1a includes, as the answer detecting section, a viewing direction detecting section that detects a viewing direction of the subject 2.

Known technologies for detecting a viewing direction may be used for the viewing direction detecting section. For example, the viewing direction detecting section may be constituted by a near infrared LED, an imaging device, and the processing section 31. The near infrared LED irradiates the eyes of the subject 2 with near infrared rays. The imaging device images the eyes of the subject 2. The processing section 31 analyzes an image or data captured by the imaging device and generates data indicating positions of pupils (a viewing direction) of the subject 2. Through the above, the processing section 31 can generate an image corresponding to data indicating a viewing direction of the subject 2 and cause for example the display section 35 to display the generated image. Consequently, a user of the dual-task performing ability evaluation system 1a can check the viewing direction (data indicating the viewing direction) of the subject 2 and calculate an answer score of the subject 2. Data indicating a viewing direction may be referred to below as viewing direction data.

In a configuration in which the dual-task performing ability evaluation system 1a includes the viewing direction detecting section, the processing section 31 may function as an analyzing section and calculate an answer score of the subject 2. That is, the processing section 31 may analyze the viewing direction data and generate data indicating an answer score.

When the intelligence task is a task of pressing a switch or two switches according to instructions of an examiner, the dual-task performing ability evaluation system 1a includes a switch or two switches as the answer detecting section 4.

In a configuration in which the answer detecting section 4 includes a switch, the answer detecting section 4 generates a signal that indicates whether or not the subject 2 pressed the switch. Based on output of the answer detecting section 4, the processing section 31 generates for example data that indicates timings at which the subject 2 pressed the switch. Specifically, the processing section 31 generates data that indicates timings at which the subject 2 pressed the switch in time series. Through the above, the processing section 31 can generate an image that indicates the timings at which the subject 2 pressed the switch in time series and cause for example the display section 35 to display the generated image. Consequently, a user of the dual-task performing ability evaluation system 1a can check the timings at which the subject 2 pressed the switch (signals indicating whether or not the switch was pressed) and calculate an answer score of the subject 2.

In the configuration in which the dual-task performing ability evaluation system 1a includes a switch, the processing section 31 may function as an analyzing section and calculate an answer score of the subject 2. That is, the processing section 31 may analyze signals indicating whether or not the subject 2 pressed the switch and generate data indicating an answer score.

In a configuration in which the answer detecting section 4 includes two switches, the answer detecting section 4 generates for example a signal that indicates which of the switches the subject 2 pressed. Based on output of the answer detecting section 4, the processing section 31 generates for example data that indicates which of the switches the subject 2 pressed in time series. Through the above, the processing section 31 can generate an image that indicates which of the switches the subject 2 pressed in time series and cause for example the display section 35 to display the generated image. Consequently, a user of the dual-task performing ability evaluation system 1a can check which of the switches the subject 2 pressed (signals indicating which of the switches was pressed) and calculate an answer score of the subject 2.

In the configuration in which the dual-task performing ability evaluation system 1a includes two switches, the processing section 31 may function as an analyzing section and calculate an answer score of the subject 2. That is, the processing section 31 may analyze signals indicating which of the switches the subject 2 pressed and generate data indicating an answer score.

### Second Embodiment

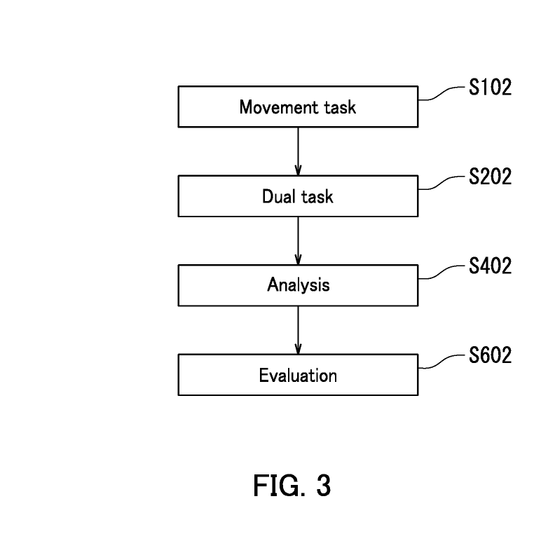

Next, a dual-task performing ability evaluation method according to a second embodiment, a dual-task performing ability evaluation system 1b, and a method for evaluating dual-task performing ability by using the dual-task performing ability evaluation system 1b will be explained with reference to FIGS. 3 to 6. Matter different from the first embodiment will be explained and explanation of the same matter as the first embodiment will be omitted. The second embodiment differs from the first embodiment in that dual-task performing ability is evaluated based on a state of movement of a subject performing a dual task.

FIG. 3 is a diagram illustrating a flow of the dual-task performing ability evaluation method according to the second embodiment. As illustrated in FIG. 3, the dual-task performing ability evaluation method according to the second embodiment includes a movement task step S102, a dual task step S202, an analysis step S402, and an evaluation step S602.

In the movement task step S102, a subject performs only a movement task that requires the subject to perform specific movement for a specific period of time. Further, in the movement task step S102, motion of the subject is detected.

In the dual task step S202, the subject performs a dual task for a specific period of time. Further, in the dual task step S202, motion of the subject is detected. The subject performs the same movement task in the movement task step S102 and the dual task step S202. The order of the movement task step S102 and the dual task step S202 may be reversed.

In the analysis step S402, a state of movement of the subject while performing the movement task step S102 and a state of movement of the subject while performing the dual task step S202 are analyzed. Specifically, in the second embodiment, a specific evaluation item that indicates a state of movement of the subject is evaluated. Further, it is determined with respect to the specific evaluation item, whether or not a state of movement of the subject is consistent between the movement task step S102 and the dual task step S202.

In the evaluation step S602, dual-task performing ability of the subject is evaluated based on a result of the analysis (result of the determination) obtained in the analysis step S402. In the evaluation step S602, evaluation data indicating the dual-task performing ability can be generated.

The dual-task performing ability evaluated in the evaluation step S602 corresponds to a commonly used intelligence evaluation scale such as an MMSE score or a Hasegawa's Dementia Scale. In the second embodiment, it is determined in the evaluation step S602 whether or not an intelligence evaluation scale of the subject is at least a specific value. The specific value may change according to difficulty levels of the movement task and the intelligence task.

For example, when walking in place for one minute is employed as the movement task and recitation of expressions starting from a syllable “ka” is employed as the intelligence task, whether or not an MMSE score of the subject is at least 27 is determined depending on whether or not a stepping rate (specific evaluation item) is consistent between the movement task step S102 and the dual task step S202. That is, when the stepping rate is consistent between the movement task step S102 and the dual task step S202, the MMSE score of the subject is determined to be at least 27. Also, whether or not the MMSE score of the subject is at least 24 is determined depending on whether or not a step width while walking in place (specific evaluation item) is consistent between the movement task step S102 and the dual task step S202. That is, when the step width while walking in place is consistent between the movement task step S102 and the dual task step S202, the MMSE score of the subject is determined to be at least 24. The step width refers to a width (distance) between right and left feet (heels).

Next, the dual-task performing ability evaluation system 1b according to the second embodiment will be explained with reference to FIG. 4. FIG. 4 is a block diagram illustrating configuration of the dual-task performing ability evaluation system 1b. The dual-task performing ability evaluation method illustrated in FIG. 3 is performed using the dual-task performing ability evaluation system 1b. As illustrated in FIG. 4, the dual-task performing ability evaluation system 1b includes the information processing device 3 and a motion detecting section 5.

The information processing device 3 includes the processing section 31, the storage section 32, the input section 34, and the display section 35. Similarly to the first embodiment, the information processing device 3 may include for example a personal computer. Alternatively, the information processing device 3 may include a device that can process information such as a smartphone or a tablet terminal. The motion detecting section 5 may be incorporated in the information processing device 3.

The motion detecting section 5 detects motion of the subject 2. In the second embodiment, the motion detecting section 5 includes an imaging section 51 and a motion capture section 52. That is, the motion detecting section 5 is a motion capture device.

The imaging section 51 images the subject 2. The imaging section 51 may include for example a CCD image sensor, a CMOS image sensor, or a laser range scanner (range sensor). The motion capture section 52 detects motion of the subject 2 imaged by the imaging section 51. Specifically, the motion capture section 52 converts movement of parts of the subject 2 to vector data and generates motion capture data reflecting the movement of the parts of the subject 2 (motion of the subject 2).

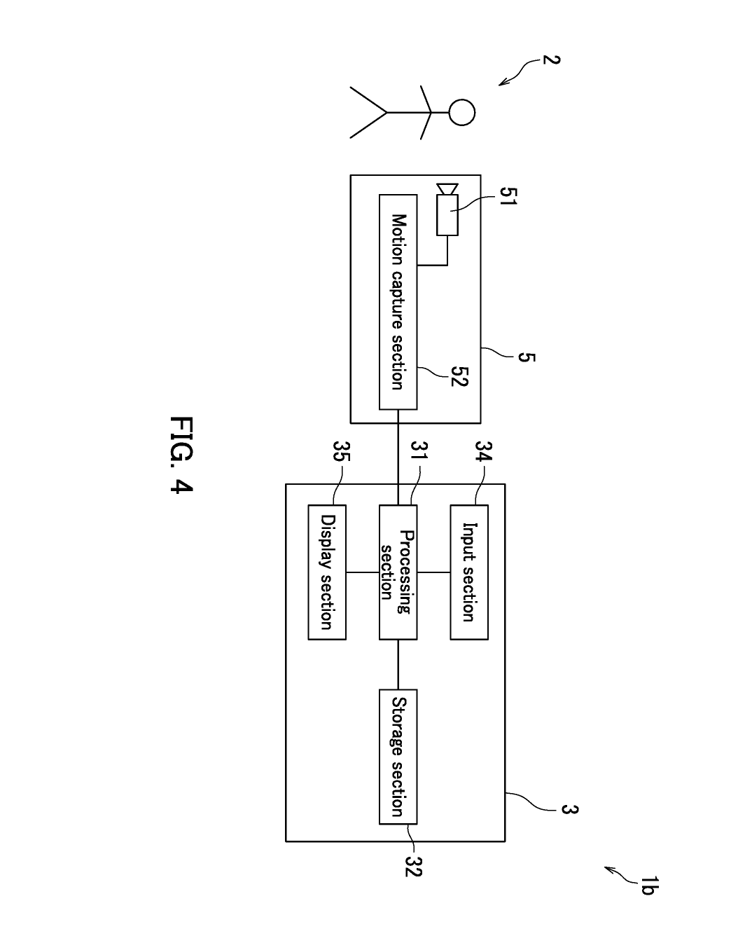

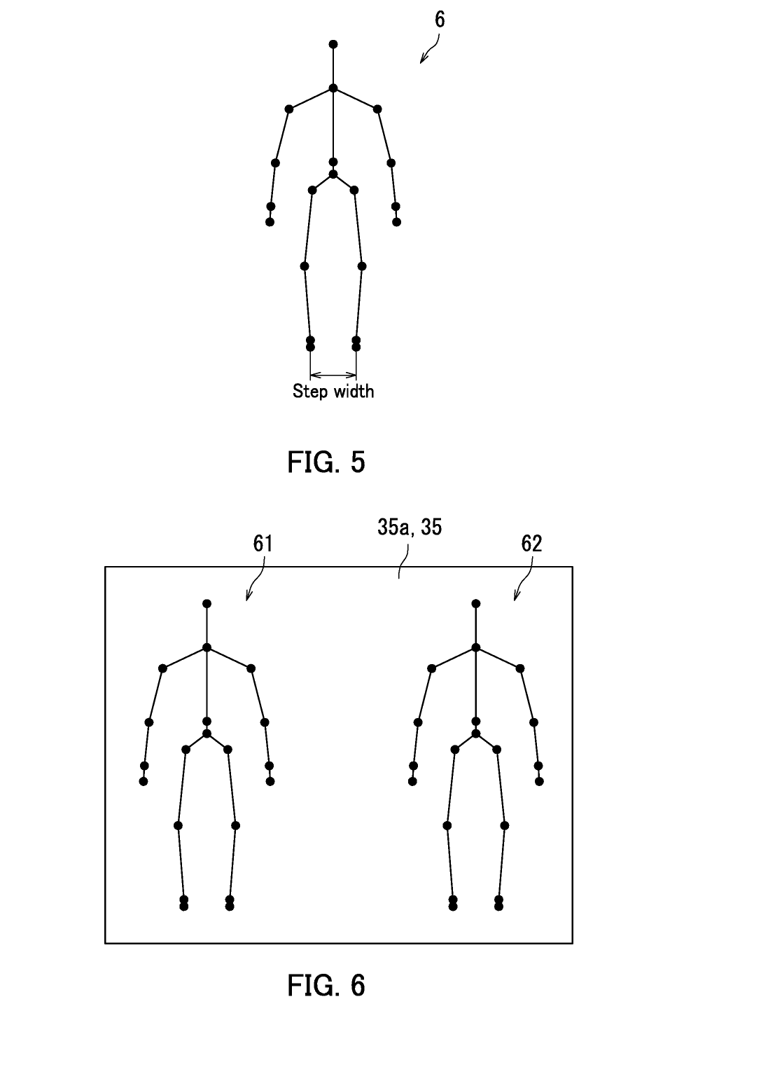

Based on output (the motion capture data) of the motion detecting section 5, the processing section 31 generates data indicating a human body skeleton model that moves along with the motion of the subject 2 imaged by the imaging section 51. FIG. 5 is a diagram illustrating a human body skeleton model. As illustrated in FIG. 5, a human body skeleton model 6 represents the structure of a human body as a link structure (tree structure).

Next, the method for evaluating dual-task performing ability of the subject 2 by using the dual-task performing ability evaluation system 1b will be explained. Specifically, a method for determining an intelligence evaluation scale of the subject 2 will be explained.

In determining the intelligence evaluation scale of the subject 2, a user initially images the subject 2 performing only the movement task for a specific period of time, by using the imaging section 51. In response, the processing section 31 generates data indicating a first human body skeleton model that moves along with motion of the subject 2 performing the movement task only. The first human body skeleton model data is stored in the storage section 32.

Next, the user images the subject 2 performing the dual task for a specific period of time, by using the imaging section 51. In response, the processing section 31 generates data indicating a second human body skeleton model that moves along with motion of the subject 2 performing the dual task. The second human body skeleton model data is stored in the storage section 32.

Next, the user operates the input section 34 to cause the processing section 31 to generate composite data combining the first human body skeleton model data and the second human body skeleton model data. Then, the user operates the input section 34 to cause the display section 35 to display images corresponding to the composite data.

FIG. 6 is a diagram illustrating human body skeleton models displayed on the display section 35. Specifically, FIG. 6 illustrates a state in which images of two human body skeleton models 61 and 62 are displayed on the display section 35. In the second embodiment, the two human body skeleton models 61 and 62 correspond to the first human body skeleton model data and the second human body skeleton model data, respectively.

As illustrated in FIG. 6, the images of the two human body skeleton models 61 and 62 are simultaneously displayed on a screen 35a of the display section 35. Through the above, the user can recognize the first human body skeleton model data corresponding to the subject 2 performing only the movement task and the second human body skeleton model data corresponding to the subject 2 performing the dual task.

Based on the first and second human body skeleton model data, the user determines with respect to a specific evaluation item, whether or not a state of movement of the subject 2 performing only the movement task is consistent with a state of movement of the subject 2 performing the dual task.

Next, the user operates the input section 34 to input data indicating a result of the determination (an analysis result). In the second embodiment, the storage section 32 stores therein an evaluation table that associates the specific evaluation item with the intelligence evaluation scale. The processing section 31 determines whether or not the intelligence evaluation scale of the subject 2 is at least a specific value based on the data indicating the result of the determination and the evaluation table.

Also, the processing section 31 generates an evaluation result image that indicates whether or not the intelligence evaluation scale of the subject 2 is at least a specific value, and causes the display section 35 to display the generated evaluation result image. Thus, the processing section 31 generates data indicating whether or not the intelligence evaluation scale of the subject 2 is at least a specific value, as evaluation data indicating the dual-task performing ability of the subject 2. As such, the processing section 31 functions as an evaluation data generating section that generates the evaluation data.

Note that the user may determine whether or not the intelligence evaluation scale of the subject 2 is at least a specific value based on the first and second human body skeleton model data.

As described above, according to the second embodiment, dual-task performing ability corresponding to an intelligence evaluation scale can be evaluated by having a subject perform a movement task (single task) and a dual task. Therefore, a degree of dementia can be evaluated more easily in a shorter time as compared with a case where dementia is diagnosed through a medical interview such as an MMSE or an intelligence test.

Also, the system for evaluating the dual-task performing ability corresponding to an intelligence evaluation scale can be constituted by the information processing device 3 such as a personal computer and the motion detecting section 5 such as a motion capture device. Therefore, the system can be simplified as compared with a case where a mental disorder such as dementia is diagnosed by using nerve image processing technologies such as magnetic resonance imaging and computer tomographic imaging.

Further, according to the second embodiment, physical functions (motor functions) of the subject can be evaluated based on an achievement level of the movement task (movement assignment). In other words, physical ability can be evaluated. Also, the subject 2 can individually evaluate physical ability together with the dual-task performing ability corresponding to an intelligence evaluation scale by using the dual-task performing ability evaluation system 1b. Therefore, the subject 2 can keep a daily record of evaluation of physical ability together with a degree of a mental disorder such as dementia. Through the above, for example worsening trends of the mental disorder such as dementia and physical functions can be detected early to conduct appropriate rehabilitation and medical treatment. Consequently, progress of the mental disorder such as dementia and decline of physical functions can be suppressed or prevented.

In the second embodiment, the dual-task performing ability evaluation system 1b is constituted by the information processing device 3 such as a personal computer and the motion capture device (motion detecting section 5). However, elements of configuration of the dual-task performing ability evaluation system 1b are not limited to those described above. For example, devices that can process information such as a smartphone and a tablet terminal may be used as an element that implements a function of the evaluation data generating section.

Also, the images of the human body skeleton models are displayed on the display section 35 (data output section) included in the information processing device 3. However, the images of the human body skeleton models may be displayed for example on a display (data output section) that is connected as an external device to the information processing device 3.

Also, in the above-explained embodiment, the user analyzes the human body skeleton model data. However, the processing section 31 may function as an analyzing section. That is, the processing section 31 may analyze the motion capture data or the human body skeleton model data and determine with respect to a specific evaluation item, whether or not a state of movement of the subject 2 is consistent between the movement task step and the dual task step.

Also, in the above-explained embodiment, the dual-task performing ability evaluation system 1b includes the motion capture device as the motion detecting section 5. However, the dual-task performing ability evaluation system 1b is not limited to the above configuration. For example, the dual-task performing ability evaluation system 1b may include a vibration detecting sensor, an imaging device, or a mat-like pressure sensitive switch, as the motion detecting section 5.

In a configuration in which a vibration detecting sensor that detects vibration of the subject 2 is used as the motion detecting section 5, the motion detecting section 5 generates signals indicating timings at which vibration was generated by movement of the subject 2.

Based on output of the motion detecting section 5, the processing section 31 generates for example data indicating the timings at which vibration was generated in time series. Through the above, the processing section 31 can cause for example the display section 35 to display an image indicating the timings at which vibration was generated by movement of the subject 2 in time series. Consequently, a user of the dual-task performing ability evaluation system 1b can analyze a state of movement of the subject 2. For example, when walking in place is employed as the movement task, the user can recognize a stepping rate of the subject 2 by checking the image indicating the timings at which vibration was generated in time series (signals indicating the timings at which vibration was generated).

In the configuration in which the dual-task performing ability evaluation system 1b includes the vibration detecting sensor, the processing section 31 may function as an analyzing section. That is, the processing section 31 may analyze a state of movement of the subject 2 based on output of the vibration detecting sensor (motion detecting section 5). For example, when walking in place is employed as the movement task, the processing section 31 can measure a stepping rate based on output of the vibration detecting sensor.

A sensor included in a smartphone may for example be used as the vibration detecting sensor. Thus, data indicating a state of movement of the subject 2 can be obtained by making the subject 2 have a smartphone while performing the movement task and the dual task.

In a configuration in which an imaging device is used as the motion detecting section 5, the motion detecting section 5 generates image data of the subject 2 imaged by the imaging device. In the above configuration, the processing section 31 causes for example the display section 35 to display an image of the subject 2 imaged by the imaging device. Through the above, a user of the dual-task performing ability evaluation system 1b can analyze a state of movement of the subject 2 by observing the image of the imaged subject 2.

In a configuration in which a mat-like pressure sensitive switch is used as the motion detecting section 5, the motion detecting section 5 generates signals indicating timings at which the subject 2 stepped on a mat. Based on output of the motion detecting section 5, the processing section 31 generates for example data indicating the timings at which the subject 2 stepped on the mat in time series. Through the above, the processing section 31 can cause for example the display section 35 to display an image indicating the timings at which the subject 2 stepped on the mat in time series. Consequently, a user of the dual-task performing ability evaluation system 1b can analyze a state of movement of the subject 2 (signals indicating the timings at which the subject 2 stepped on the mat).

In the configuration in which the dual-task performing ability evaluation system 1b includes the mat-like pressure sensitive switch, the processing section 31 may function as an analyzing section. That is, the processing section 31 may analyze a state of movement of the subject 2 based on output of the mat-like pressure sensitive switch (motion detecting section 5). For example, when walking in place is employed as the movement task, the processing section 31 can measure a stepping rate based on output of the mat-like pressure sensitive switch.

### Third Embodiment

Next, a dual-task performing ability evaluation method according to a third embodiment, the dual-task performing ability evaluation system 1b, and a method for evaluating dual-task performing ability by using the dual-task performing ability evaluation system 1b will be explained with reference to FIGS. 4, 7, and 8. Matter different from the first and second embodiments will be explained and explanation of the same matter as the first and second embodiments will be omitted. The third embodiment differs from the first and second embodiments in that a subject performs a movement task and plural kinds of dual tasks.

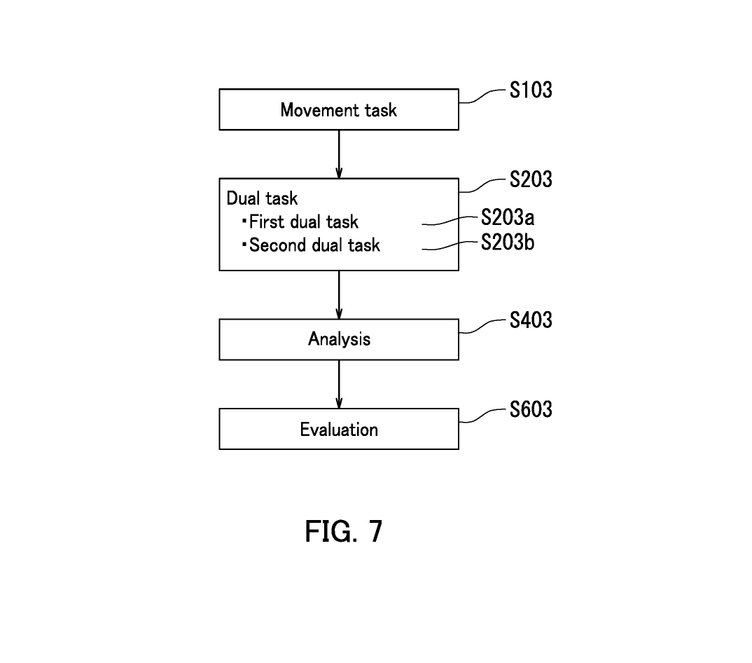

FIG. 7 is a diagram illustrating a flow of the dual-task performing ability evaluation method according to the third embodiment. As illustrated in FIG. 7, the dual-task performing ability evaluation method according to the third embodiment includes a movement task step S103, a dual task step S203, an analysis step S403, and an evaluation step S603.

In the movement task step S103, a subject performs only a movement task for a specific period of time. Further, in the movement task step S103, motion of the subject performing the movement task is detected.

The dual task step S203 includes a first dual task step S203a and a second dual task step S203b. In the first dual task step S203a, the subject performs a first dual task for a specific period of time. Further, in the first dual task step S203a, motion of the subject performing the first dual task is detected. In the second dual task step S203b, the subject performs a second dual task for a specific period of time. Further, in the second dual task step S203b, motion of the subject performing the second dual task is detected.

Difficulty levels of intelligence tasks included in the first dual task step S203a and the second dual task step S203b are different from each other. Specifically, a difficulty level of an intelligence task included in the second dual task step S203b is higher than that of an intelligence task included in the first dual task step S203a. The subject performs the same movement task in the movement task step S103 and the dual task step S203 (the first dual task step S203a and the second dual task step S203b). The order of the movement task step S103 and the dual task step S203 may be reversed.

In the analysis step S403, a state of movement of the subject while performing the movement task step S103, a state of movement of the subject while performing the first dual task step S203a, and a state of movement of the subject while performing the second dual task step S203b are analyzed. Specifically, in the third embodiment, plural evaluation items each indicate a state of movement of the subject are evaluated. Further, it is determined whether or not evaluation results of at least two of the plural evaluation items become lower in order of the movement task step S103 (movement task), the first dual task step S203a (first dual task), and the second dual task step S203b (second dual task). The plural evaluation items may include for example items for evaluating magnitude of movement, items for evaluating agility, and items for evaluating stability.

In the evaluation step S603, dual-task performing ability of the subject is evaluated based on a result of the analysis (a result of the determination) obtained in the analysis step S403. In the evaluation step S603, evaluation data indicating the dual-task performing ability can be generated.

The dual-task performing ability evaluated in the evaluation step S603 corresponds to a commonly used intelligence evaluation scale such as an MMSE score or a Hasegawa's Dementia Scale. In the third embodiment, it is determined in the evaluation step S603 whether or not an intelligence evaluation scale of the subject is a specific value. The specific value may change according to difficulty levels of the movement task and the intelligence tasks.

For example, when walking in place for one minute is employed as the movement task and serial subtraction is employed as the intelligence task of the first dual task while recitation of expressions starting from a syllable “ka” is employed as the intelligence task of the second dual task, it is determined whether or not an MMSE score of the subject is 27 or 26. That is, when evaluation results of at least two of plural evaluation items become lower in order of the movement task step S103, the first dual task step S203a, and the second dual task step S203b, the MMSE score of the subject is determined to be 26 or 27. When the subject walks in place, for example a height of a raised foot, a height of a knee, and a length of arm swing can be evaluated as magnitude of movement. Further, for example a stepping rate (cycle) can be evaluated as agility. Also, for example shortness of a step width and slightness of wobbling of the upper half of the body can be evaluated as stability.

Next, the dual-task performing ability evaluation system 1b according to the third embodiment will be explained with reference to FIG. 4. The dual-task performing ability evaluation method illustrated in FIG. 7 is performed using the dual-task performing ability evaluation system 1b.

In the third embodiment, the imaging section 51 images motion of the subject 2 performing only the movement task, motion of the subject 2 performing the first dual task, and motion of the subject 2 performing the second dual task. Also, the motion capture section 52 generates motion capture data reflecting the motion of the subject 2 performing only the movement task. Further, the motion capture section 52 generates motion capture data reflecting the motion of the subject 2 performing the first dual task and motion capture data reflecting the motion of the subject 2 performing the second dual task.

Based on output (the motion capture data) of the motion detecting section 5, the processing section 31 generates data indicating a third human body skeleton model, data indicating a fourth human body skeleton model, and data indicating a fifth human body skeleton model. The third human body skeleton model moves along with the motion of the subject 2 performing only the movement task. The fourth human body skeleton model moves along with the motion of the subject 2 performing the first dual task. The fifth human body skeleton model moves along with the motion of the subject 2 performing the second dual task. Further, the processing section 31 generates composite data combining the third human body skeleton model data, the fourth human body skeleton model data, and the fifth human body skeleton model data.

Next, the method for evaluating dual-task performing ability of the subject 2 by using the dual-task performing ability evaluation system 1b will be explained. Specifically, a method for determining an intelligence evaluation scale of the subject 2 will be explained.

In determining the intelligence evaluation scale of the subject 2, a user initially images the subject 2 performing only the movement task for a specific period of time, by using the imaging section 51. In response, the processing section 31 generates the third human body skeleton model data. The third human body skeleton model data is stored in the storage section 32.

Next, the user images the subject 2 performing the first dual task for a specific period of time, by using the imaging section 51. In response, the processing section 31 generates the fourth human body skeleton model data. The fourth human body skeleton model data is stored in the storage section 32.

Next, the user images the subject 2 performing the second dual task for a specific period of time, by using the imaging section 51. In response, the processing section 31 generates the fifth human body skeleton model data. The fifth human body skeleton model data is stored in the storage section 32.

Next, the user operates the input section 34 to cause the processing section 31 to generate composite data combining the third human body skeleton model data, the fourth human body skeleton model data, and the fifth human body skeleton model data. Then, the user operates the input section 34 to cause the display section 35 to display images corresponding to the composite data.

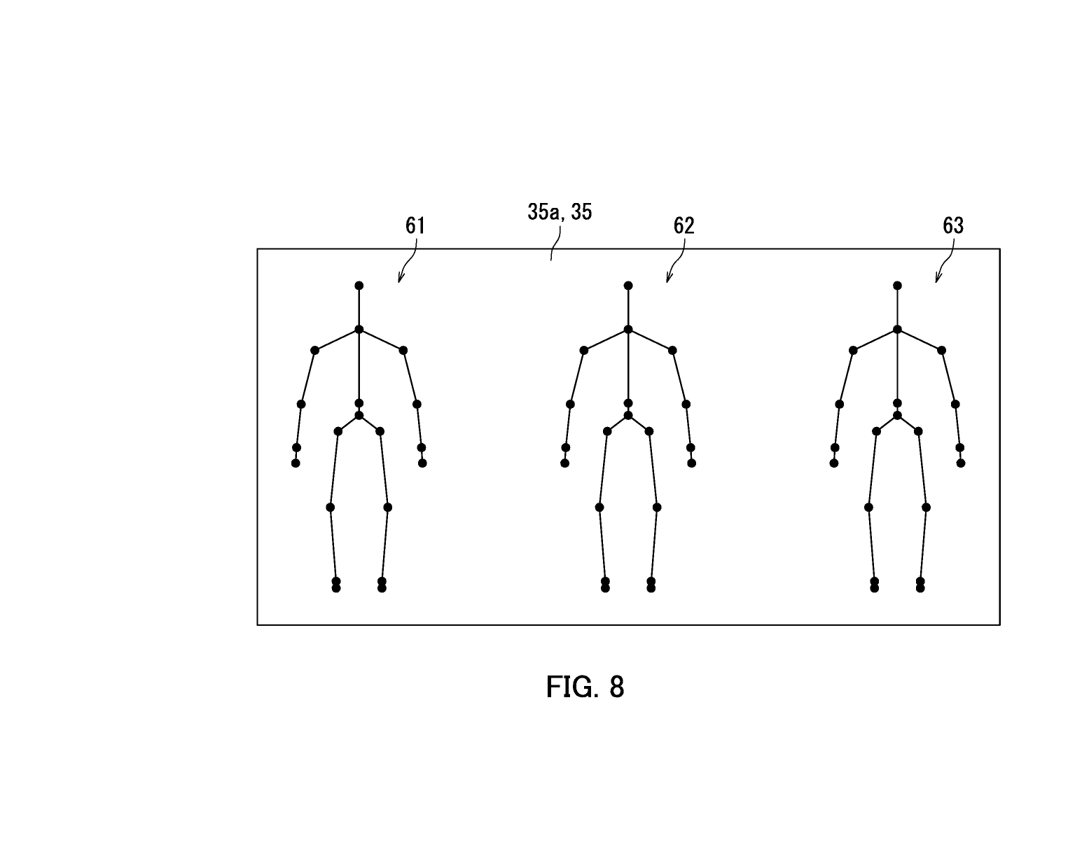

FIG. 8 is a diagram illustrating human body skeleton models displayed on the display section 35. Specifically, FIG. 8 illustrates a state in which images of three human body skeleton models 61, 62, and 63 are displayed on the display section 35. In the third embodiment, the three human body skeleton models 61, 62, and 63 correspond to the third human body skeleton model data, the fourth human body skeleton model data, and the fifth human body skeleton model data, respectively.

As illustrated in FIG. 8, the images of the three human body skeleton models 61, 62, and 63 are simultaneously displayed on the screen 35a of the display section 35. Through the above, the user can recognize the third human body skeleton model data corresponding to the subject 2 performing only the movement task, the fourth human body skeleton model data corresponding to the subject 2 performing the first dual task, and the fifth human body skeleton model data corresponding to the subject 2 performing the second dual task.

Based on the third, fourth, and fifth human body skeleton model data, the user evaluates states of movement of the subject with respect to plural evaluation items. Specifically, the user determines whether or not evaluation results of at least two of the plural evaluation items become lower in order of the third human body skeleton model data (the movement task), the fourth human body skeleton model data (the first dual task), and the fifth human body skeleton model data (the second dual task). The plural evaluation items include for example a height of a raised foot, a height of a knee, a length of arm swing, a stepping rate, shortness of a step width, and slightness of wobbling of the upper half of the body.

Next, the user operates the input section 34 to input data indicating a result of the determination (an analysis result). The processing section 31 determines whether or not the intelligence evaluation scale of the subject 2 is a specific value based on the data indicating the result of the determination. For example, when walking in place for one minute is employed as the movement task and serial subtraction is employed as the intelligence task of the first dual task while recitation of expressions starting from a syllable “ka” is employed as the intelligence task of the second dual task, it can be determined whether or not the MMSE score of the subject 2 is 26 or 27.

Also, the processing section 31 generates an evaluation result image that indicates whether or not the intelligence evaluation scale of the subject 2 is a specific value, and causes the display section 35 to display the generated evaluation result image. Thus, the processing section 31 generates data indicating whether or not the intelligence evaluation scale of the subject 2 is a specific value, as evaluation data indicating the dual-task performing ability of the subject 2. As such, the processing section 31 functions as an evaluation data generating section that generates the evaluation data.

Note that the user may determine whether or not the intelligence evaluation scale of the subject 2 is a specific value based on the third, fourth, and fifth human body skeleton model data.

As described above, according to the third embodiment, dual-task performing ability corresponding to an intelligence evaluation scale can be evaluated by having a subject perform a movement task (single task) and two kinds (an example of plural kinds) of dual tasks. Therefore, a degree of dementia can be evaluated more easily in a shorter time as compared with a case where dementia is diagnosed through a medical interview such as an MMSE or an intelligence test.

Also, according to the third embodiment, the system can be simplified similarly to the second embodiment as compared with a case where a mental disorder such as dementia is diagnosed by using nerve image processing technologies.

Further, according to the third embodiment, physical functions (motor functions) of the subject can be evaluated based on an achievement level of the movement task (movement assignment) similarly to the second embodiment. Also, the subject 2 can individually evaluate physical ability together with a degree of a mental disorder such as dementia by using the dual-task performing ability evaluation system 1b.

In the third embodiment, the dual-task performing ability evaluation system 1b is constituted by the information processing device 3 such as a personal computer and the motion capture device (motion detecting section 5) similarly to the second embodiment. However, elements of configuration of the dual-task performing ability evaluation system 1b are not limited to those described above. For example, devices that can process information such as a smartphone and a tablet terminal may be used as an element that implements the evaluation data generating section.

Also, the images of the human body skeleton models are displayed on the display section 35 (data output section) included in the information processing device 3. However, the images of the human body skeleton models may be displayed for example on a display (data output section) that is connected as an external device to the information processing device 3.

Also, in the above-explained embodiment, the user analyzes the human body skeleton model data. However, the processing section 31 may function as an analyzing section. That is, the processing section 31 may analyze the motion capture data or the human body skeleton model data and determine whether or not evaluation results of at least two evaluation items become lower in order of the movement task step (the movement task), the first dual task step (the first dual task), and the second dual task step (the second dual task).

Also, in the above-explained embodiment, the motion detecting section 5 captures (detects) movement of parts of the subject 2 by a motion capture technology. However, the dual-task performing ability evaluation system 1b is not limited to the above configuration. For example, the dual-task performing ability evaluation system 1b may include an imaging device as the motion detecting section 5.

In a configuration in which an imaging device is used as the motion detecting section 5, the motion detecting section 5 generates image data of the subject 2 imaged by the imaging device. In the above configuration, the processing section 31 causes for example the display section 35 to display an image of the subject 2 imaged by the imaging device. Through the above, a user of the dual-task performing ability evaluation system 1b can analyze a state of movement of the subject 2 by observing the image of the imaged subject 2.

### Fourth Embodiment

Next, a dual-task performing ability evaluation method according to a fourth embodiment, the dual-task performing ability evaluation system 1b, and a method for evaluating dual-task performing ability by using the dual-task performing ability evaluation system 1b will be explained with reference to FIGS. 4, 6, and 9. Matter different from the first through third embodiments will be explained and explanation of the same matter as the first through third embodiments will be omitted. The fourth embodiment differs from the first through third embodiments in that a subject performs only plural kinds of dual tasks.

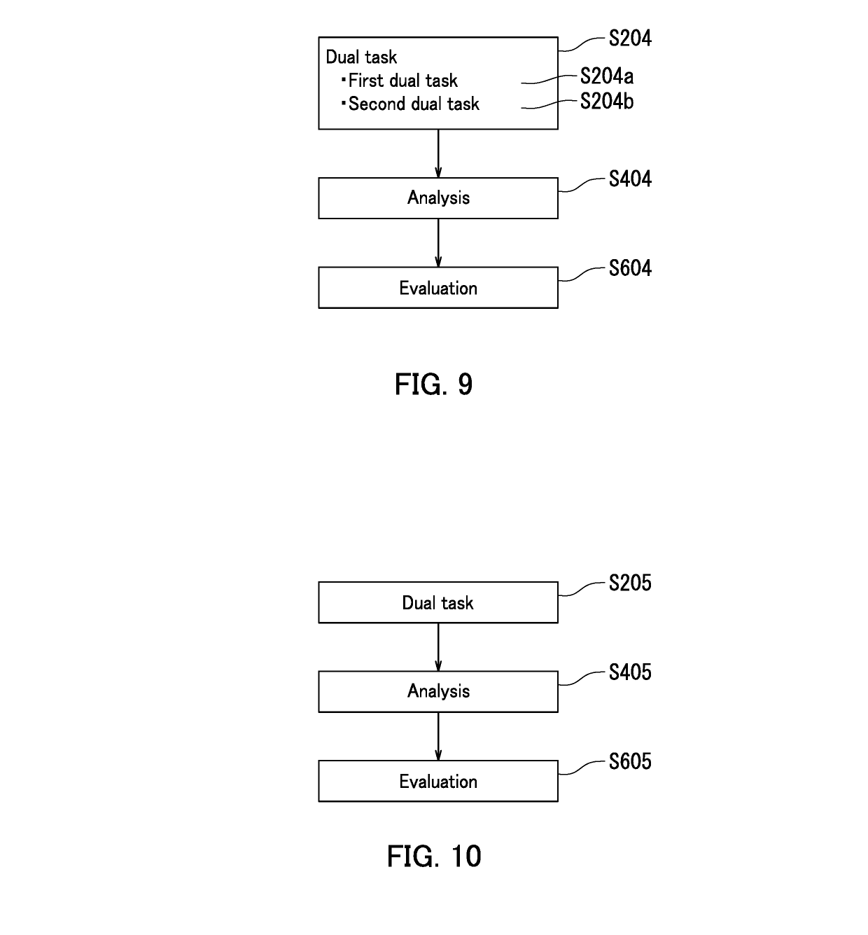

FIG. 9 is a diagram illustrating a flow of the dual-task performing ability evaluation method according to the fourth embodiment. As illustrated in FIG. 9, the dual-task performing ability evaluation method according to the fourth embodiment includes a dual task step S204, an analysis step S404, and an evaluation step S604.

The dual task step S204 includes a first dual task step S204a and a second dual task step S204b. In the first dual task step S204a, a subject performs a first dual task for a specific period of time. In the first dual task step S204a, motion of the subject performing the first dual task is detected. In the second dual task step S204b, the subject performs a second dual task for a specific period of time. In the second dual task step S204b, motion of the subject performing the second dual task is detected.

Difficulty levels of intelligence tasks included in the first dual task step S204a and the second dual task step S204b are different from each other. Specifically, a difficulty level of an intelligence task included in the second dual task step S204b is higher than that of an intelligence task included in the first dual task step S204a. The subject performs the same movement task in the first dual task step S204a and the second dual task step S204b.

In the analysis step S404, a state of movement of the subject while performing the first dual task step S204a and a state of movement of the subject while performing the second dual task step S204b are analyzed. Specifically, in the fourth embodiment, continuity of movement of the subject (whether or not movement of the subject continued for a specific period of time) is determined. That is, it is determined whether or not the subject stopped the movement task (specific movement) while performing the first dual task or the second dual task.

In the evaluation step S604, dual-task performing ability of the subject is evaluated based on a result of the analysis (a result of the determination) obtained in the analysis step S404. In the evaluation step S604, evaluation data indicating the dual-task performing ability can be generated.

The dual-task performing ability evaluated in the evaluation step S604 corresponds to a commonly used intelligence evaluation scale such as an MMSE score or a Hasegawa's Dementia Scale. In the fourth embodiment, it is determined in the evaluation step S604 whether or not an intelligence evaluation scale of the subject is no greater than a specific value. The specific value may change according to difficulty levels of the movement task and the intelligence tasks.

For example, when walking in place for one minute is employed as the movement task and serial subtraction is employed as the intelligence task of the first dual task while recitation of expressions starting from a syllable “ka” is employed as the intelligence task of the second dual task, it is determined whether or not an MMSE score of the subject is no greater than 24. That is, when the subject stopped the movement task (the specific movement) while performing the first dual task or the second dual task, the MMSE score of the subject is determined to be no greater than 24.

Next, the dual-task performing ability evaluation system 1b according to the fourth embodiment will be explained with reference to FIG. 4. The dual-task performing ability evaluation method illustrated in FIG. 9 is performed using the dual-task performing ability evaluation system 1b.

In the fourth embodiment, the imaging section 51 images motion of the subject 2 performing the first dual task and motion of the subject 2 performing the second dual task. Also, the motion capture section 52 generates motion capture data reflecting the motion of the subject 2 performing the first dual task and motion capture data reflecting the motion of the subject 2 performing the second dual task.

Based on output (the motion capture data) of the motion detecting section 5, the processing section 31 generates data indicating a sixth human body skeleton model and data indicating a seventh human body skeleton model. The sixth human body skeleton model moves along with the motion of the subject 2 performing the first dual task. The seventh human body skeleton model moves along with the motion of the subject 2 performing the second dual task. Further, the processing section 31 generates composite data combining the sixth human body skeleton model data and the seventh human body skeleton model data.

Next, the method for evaluating dual-task performing ability of the subject 2 by using the dual-task performing ability evaluation system 1b will be explained. Specifically, a method for determining an intelligence evaluation scale of the subject 2 will be explained.

In determining the intelligence evaluation scale of the subject 2, a user initially images the subject 2 performing the first dual task for a specific period of time, by using the imaging section 51. In response, the sixth human body skeleton model data is generated. The sixth human body skeleton model data is stored in the storage section 32.

Next, the user images the subject 2 performing the second dual task for a specific period of time, by using the imaging section 51. In response, the seventh human body skeleton model data is generated. The seventh human body skeleton model data is stored in the storage section 32.

Next, the user operates the input section 34 to cause the processing section 31 to generate composite data combining the sixth human body skeleton model data and the seventh human body skeleton model data. Then, the user operates the input section 34 to cause the display section 35 to display images corresponding to the composite data. As a result, images of two human body skeleton models 61 and 62 are simultaneously displayed on the screen 35a of the display section 35 as illustrated in FIG. 6.

In the fourth embodiment, the two human body skeleton models 61 and 62 correspond to the sixth human body skeleton model data and the seventh human body skeleton model data, respectively. Through the above, the user can recognize the sixth human body skeleton model data corresponding to the subject 2 performing the first dual task and the seventh human body skeleton model data corresponding to the subject 2 performing the second dual task.

Based on the sixth and seventh human body skeleton model data, the user determines whether or not the subject 2 stopped the movement task (the specific movement) while performing the first dual task or the second dual task.

Next, the user operates the input section 34 to input data indicating a result of the determination (an analysis result). The processing section 31 determines whether or not the intelligence evaluation scale of the subject 2 is no greater than a specific value based on the data indicating the result of the determination. For example, when walking in place for one minute is employed as the movement task and serial subtraction is employed as the intelligence task of the first dual task while recitation of expressions starting from a syllable “ka” is employed as the intelligence task of the second dual task, it can be determined whether or not the MMSE score of the subject is no greater than 24.

Also, the processing section 31 generates an evaluation result image that indicates whether or not the intelligence evaluation scale of the subject 2 is no greater than a specific value, and causes the display section 35 to display the generated evaluation result image. Thus, the processing section 31 generates data indicating whether or not the intelligence evaluation scale of the subject 2 is no greater than a specific value, as evaluation data indicating the dual-task performing ability of the subject 2. As such, the processing section 31 functions as an evaluation data generating section that generates the evaluation data.

Note that the user may determine whether or not the intelligence evaluation scale of the subject 2 is no greater than a specific value based on the sixth and seventh human body skeleton model data.

As described above, according to the fourth embodiment, dual-task performing ability corresponding to an intelligence evaluation scale can be evaluated by having a subject perform two kinds (an example of plural kinds) of dual tasks only. Therefore, a degree of dementia can be evaluated more easily in a shorter time as compared with a case where dementia is diagnosed through a medical interview such as an MMSE or an intelligence test.

Also, according to the fourth embodiment, the system can be simplified similarly to the second and third embodiments as compared with a case where a mental disorder such as dementia is diagnosed by using nerve image processing technologies.

Further, according to the fourth embodiment, physical functions (motor functions) of the subject can be evaluated based on an achievement level of the movement task (movement assignment) similarly to the second and third embodiments. Also, the subject 2 can individually evaluate physical ability together with a degree of a mental disorder such as dementia by using the dual-task performing ability evaluation system 1b.

In the fourth embodiment explained above, the images of the two human body skeleton models 61 and 62 are displayed simultaneously with each other. However, the images of the two human body skeleton models 61 and 62 may be displayed separately from each other.

Also, in the fourth embodiment, the dual-task performing ability evaluation system 1b is constituted by the information processing device 3 such as a personal computer and the motion capture device (motion detecting section 5) similarly to the second and third embodiments. However, elements of configuration of the dual-task performing ability evaluation system 1b are not limited to those described above. For example, devices that can process information such as a smartphone and a tablet terminal may be used as an element that implements a function of the evaluation data generating section.

Also, in the above-explained embodiment, the user analyzes the human body skeleton model data. However, the processing section 31 may function as an analyzing section. That is, the processing section 31 may analyze the motion capture data or the human body skeleton model data and determine continuity of movement of the subject.

Also, in the above-explained embodiment, the dual-task performing ability evaluation system 1b includes the motion capture device as the motion detecting section 5. However, the dual-task performing ability evaluation system 1b is not limited to the above configuration. For example, the dual-task performing ability evaluation system 1b may include a vibration detecting sensor, an imaging device, or a mat-like pressure sensitive switch, as the motion detecting section 5.

In a configuration in which a vibration detecting sensor that detects vibration of the subject 2 is used as the motion detecting section 5, the motion detecting section 5 generates signals indicating timings at which vibration was generated by movement of the subject 2.

Based on output of the motion detecting section 5, the processing section 31 generates for example data indicating the timings at which vibration was generated in time series. Through the above, the processing section 31 can cause for example the display section 35 to display an image indicating the timings at which vibration was generated by movement of the subject 2 in time series. Consequently, a user of the dual-task performing ability evaluation system 1b can analyze a state of movement of the subject. That is, the user can recognize whether or not the subject stopped the movement. For example, when walking in place is employed as the movement task, the user can recognize whether or not the subject 2 stopped walking in place by checking the image indicating the timings at which vibration was generated in time series (signals indicating the timings at which vibration was generated).

In the configuration in which the dual-task performing ability evaluation system 1b includes the vibration detecting sensor, the processing section 31 may function as an analyzing section. That is, the processing section 31 may analyze a state of movement of the subject 2 based on output of the vibration detecting sensor (motion detecting section 5). For example, when walking in place is employed as the movement task, the processing section 31 can determine whether or not the subject 2 stopped walking in place based on output of the vibration detecting sensor.

A sensor included in a smartphone may for example be used as the vibration detecting sensor. Thus, data indicating continuity of movement of the subject 2 can be obtained by making the subject 2 have a smartphone while performing the dual task.

Also, in a configuration in which an imaging device is used as the motion detecting section 5, the motion detecting section 5 generates image data of the subject 2 imaged by the imaging device. In the above configuration, the processing section 31 causes for example the display section 35 to display an image of the subject 2 imaged by the imaging device. Through the above, a user of the dual-task performing ability evaluation system 1b can analyze continuity of movement of the subject 2 by observing the image of the imaged subject 2.

In a configuration in which a mat-like pressure sensitive switch is used as the motion detecting section 5, the motion detecting section 5 generates signals indicating timings at which the subject 2 stepped on a mat. Based on output of the motion detecting section 5, the processing section 31 generates for example data indicating the timings at which the subject 2 stepped on the mat in time series. Through the above, the processing section 31 can cause for example the display section 35 to display an image indicating the timings at which the subject 2 stepped on the mat in time series. Consequently, a user of the dual-task performing ability evaluation system 1b can analyze continuity of movement of the subject 2.

In the configuration in which the dual-task performing ability evaluation system 1b includes the mat-like pressure sensitive switch, the processing section 31 may function as an analyzing section. That is, the processing section 31 may analyze a state of movement of the subject 2 based on output of the mat-like pressure sensitive switch (motion detecting section 5). For example, when walking in place is employed as the movement task, the processing section 31 can determine whether or not the subject 2 stopped walking in place based on output of the mat-like pressure sensitive switch.

### Fifth Embodiment

Next, a dual-task performing ability evaluation method according to a fifth embodiment, a dual-task performing ability evaluation system 1c, and a method for evaluating dual-task performing ability by using the dual-task performing ability evaluation system 1c will be explained with reference to FIGS. 10, 11, and 12. Matter different from the first through fourth embodiments will be explained and explanation of the same matter as the first through fourth embodiments will be omitted. The fifth embodiment differs from the first through fourth embodiments in that dual-task performing ability is evaluated based on continuity of motion of a subject and continuity of answers of the subject.

FIG. 10 is a diagram illustrating a flow of the dual-task performing ability evaluation method according to the fifth embodiment. As illustrated in FIG. 10, the dual-task performing ability evaluation method according to the fifth embodiment includes a dual task step S205, an analysis step S405, and an evaluation step S605.

In the dual task step S205, a subject performs a dual task for a specific period of time. Further, in the dual task step S205, motion and answers of the subject performing the dual task are detected.

In the analysis step S405, continuity of the motion and answers of the subject performing the dual task is analyzed. Specifically, in the dual task step S205, it is determined whether or not the subject stopped a movement task (specific movement) and finished an intelligence task (answering) before the specific period of time elapsed.

In the evaluation step S605, dual-task performing ability of the subject is evaluated based on a result of the analysis (a result of the determination) obtained in the analysis step S405. In the evaluation step S605, evaluation data indicating the dual-task performing ability can be generated.

The dual-task performing ability evaluated in the evaluation step S605 corresponds to a commonly used intelligence evaluation scale such as an MMSE score or a Hasegawa's Dementia Scale. In the fifth embodiment, it is determined as the dual-task performing ability, whether or not an intelligence evaluation scale of the subject is no greater than a specific value. The specific value may change according to difficulty levels of the movement task and the intelligence task.

For example, when straight walking for one minute is employed as the movement task and recitation of expressions starting from a syllable “ka” is employed as the intelligence task, it is determined whether or not an MMSE score of the subject is no greater than 23. That is, when, while performing the dual task, the subject stopped walking motion (specific motion) and finished answering a problem that the intelligence task requires the subject to answer before one minute (the specific period of time) elapsed, the MMSE score of the subject is determined to be no greater than 23.

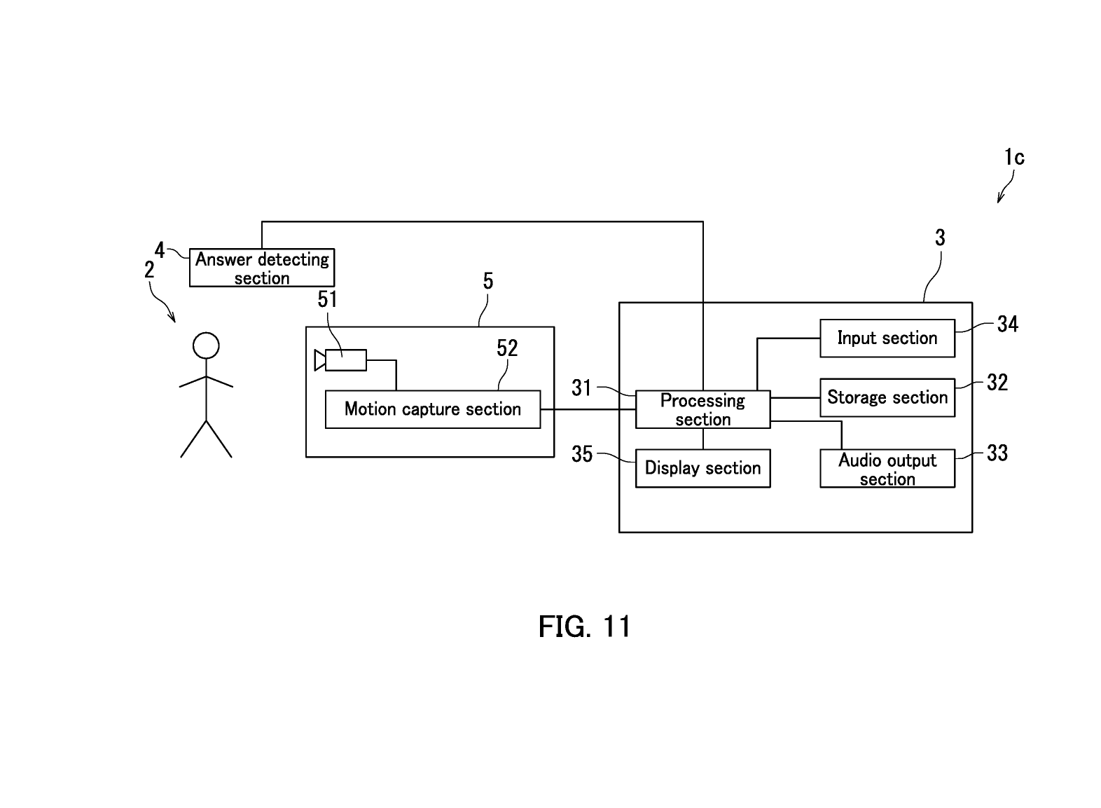

Next, the dual-task performing ability evaluation system 1c according to the fifth embodiment will be explained with reference to FIG. 11. FIG. 11 is a block diagram illustrating configuration of the dual-task performing ability evaluation system 1c. The dual-task performing ability evaluation method illustrated in FIG. 10 is performed using the dual-task performing ability evaluation system 1c.

As illustrated in FIG. 11, the dual-task performing ability evaluation system 1c includes the information processing device 3, the answer detecting section 4, and the motion detecting section 5. The information processing device 3 may include for example a personal computer. Alternatively, the information processing device 3 may include a device that can process information such as a smartphone or a tablet terminal. In the fifth embodiment, the answer detecting section 4 includes a microphone. Also, the motion detecting section 5 includes a motion capture device. At least one of the answer detecting section 4 and the motion detecting section 5 may be incorporated in the information processing device 3.

In the fifth embodiment, the processing section 31 included in the information processing device 3 generates data indicating continuity of movement of the subject 2 based on motion capture data. Specifically, in the fifth embodiment, the processing section 31 generates data indicating timings at which the subject 2 put down his feet on the ground. Also, the processing section 31 generates data indicating continuity of answers of the subject 2. Specifically, in the fifth embodiment, the processing section 31 generates data indicating timings at which the subject 2 made answers. Further, the processing section 31 generates an image corresponding to the data indicating continuity of movement of the subject 2 and an image corresponding to the data indicating continuity of answers of the subject 2.

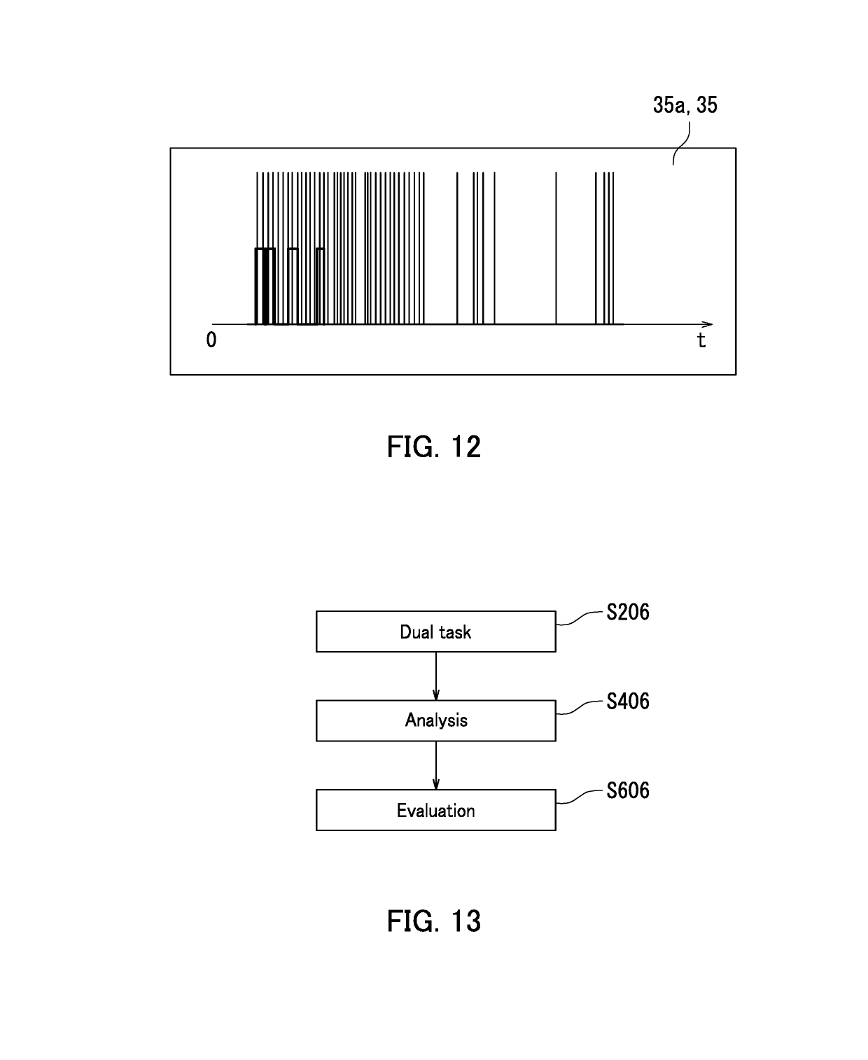

FIG. 12 is a diagram illustrating an image displayed on the display section 35. Specifically, FIG. 12 illustrates an example of an image that indicates timings at which the subject 2 put down his feet on the ground and timings at which the subject 2 made answers. The horizontal axis in FIG. 12 represents time. In FIG. 12, long thin lines indicate the timings at which the subject 2 put down his feet on the ground and short bold lines indicate the timings at which the subject 2 made answers.

Next, the method for evaluating dual-task performing ability of the subject 2 by using the dual-task performing ability evaluation system 1c will be explained. Specifically, a method for determining an intelligence evaluation scale will be explained by taking as an example, a case of determining an MMSE score of the subject 2 by employing straight walking for one minute as a movement task and recitation of expressions starting from a syllable “ka” as an intelligence task.

In determining the MMSE score (intelligence evaluation scale) of the subject 2, a user initially stores (records) in the storage section 32 audio data corresponding to voice uttered by the subject 2 performing the dual task for one minute (a specific period of time). Further, during the above period, the user images the subject 2 by using an imaging section 51. Through the above, data indicating timings at which the subject 2 performing the dual task put down his feet on the ground is generated. The generated data is stored in the storage section 32.

Next, the user operates the input section 34 to cause the audio output section 33 to output sound corresponding to the audio data stored in the storage section 32. In other words, the voice uttered by the subject 2 while performing the dual task is reproduced. Further, the user causes the display section 35 to display an elapsed time from initiation of the reproduction. The user measures timings (elapsed time from initiation of the reproduction) at which the subject 2 made answers based on the reproduced voice and the displayed elapsed time.

Next, the user operates the input section 34 to input data indicating timings of the answers. In response, the processing section 31 generates data indicating the timings at which the subject 2 made answers. Thereafter, the user operates the input section 34 to cause the processing section 31 to generate an image by combining an image indicating the timings at which the subject 2 put down his feet on the ground and an image indicating the timings at which the subject 2 made answers. Consequently, an image indicating the timings at which the subject 2 put down his feet on the ground and the timings at which the subject 2 made answers is displayed on the screen 35a of the display section 35 as illustrated in FIG. 12.

Thus, the processing section 31 generates data indicating the timings at which the subject 2 put down his feet on the ground based on motion of the subject 2 detected by the motion detecting section 5. Further, the processing section 31 generates data indicating the timings at which the subject 2 made answers based on answers of the subject 2 detected by the answer detecting section 4.

Based on the image indicating the timings at which the subject 2 put down his feet on the ground and the timings at which the subject 2 made answers (the data indicating the timings at which the subject 2 put down his feet on the ground and the data indicating the timings at which the subject 2 made answers), the user determines whether or not the subject 2 stopped the walking motion (specific motion) and finished answering before one minute (the specific period of time) elapsed while performing the dual task.

Next, the user operates the input section 34 to input data indicating a result of the determination (an analysis result). The processing section 31 determines whether or not the MMSE score of the subject 2 is no greater than 23 based on the data indicating the result of the determination.

Also, the processing section 31 generates an evaluation result image that indicates whether or not the MMSE score of the subject 2 is no greater than 23, and causes the display section 35 to display the generated evaluation result image. Thus, the processing section 31 generates data indicating whether or not the intelligence evaluation scale of the subject 2 is no greater than a specific value, as evaluation data indicating the dual-task performing ability of the subject 2. As such, the processing section 31 functions as an evaluation data generating section that generates the evaluation data.

Note that the user may determine whether or not the intelligence evaluation scale of the subject 2 is no greater than a specific value based on the timings at which the subject 2 put down his feet on the ground and the timings at which the subject 2 made answers.

As described above, according to the fifth embodiment, dual-task performing ability corresponding to an intelligence evaluation scale can be evaluated by having a subject perform a dual task only. Therefore, a degree of dementia can be evaluated more easily in a shorter time as compared with a case where dementia is diagnosed through a medical interview such as an MMSE or an intelligence test.

Also, according to the fifth embodiment, the system can be simplified similarly to the first through fourth embodiments as compared with a case where a mental disorder such as dementia is diagnosed by using nerve image processing technologies.

Further, according to the fifth embodiment, physical functions (motor functions) of the subject can be evaluated based on an achievement level of the movement task (movement assignment) similarly to the second through fourth embodiments. Also, the subject 2 can individually evaluate physical ability together with a degree of a mental disorder such as dementia by using the dual-task performing ability evaluation system 1c.

In the fifth embodiment, the dual-task performing ability evaluation system 1c is constituted by the information processing device 3 such as a personal computer, the microphone (answer detecting section 4), and the motion capture device (motion detecting section 5). However, elements of configuration of the dual-task performing ability evaluation system 1c are not limited to those described above. For example, devices such as an IC recorder and a smartphone may be used as an element that implements a function of recording audio data. Further, devices that can process information such as a smartphone and a tablet terminal may for example be used as an element that implements a function of the evaluation data generating section.

Also, a composite image combining an image indicating continuity of movement of the subject 2 (image indicating timings at which the subject 2 put down his feet on the ground) and an image indicating continuity of answers of the subject 2 (image indicating timings at which the subject 2 made answers) is displayed on the display section 35 (data output section) included in the information processing device 3. However, the composite image may be displayed for example on a display (data output section) that is connected as an external device to the information processing device 3. Also, the composite image combining an image indicating continuity of movement of the subject 2 and an image indicating continuity of answers of the subject 2 may for example be printed on a recording medium by a printer (data output section) that is connected as an external device to the information processing device 3.

Also, in the above-explained embodiment, the composite image combining an image indicating continuity of movement of the subject 2 and an image indicating continuity of answers of the subject 2 is displayed or printed. However, an image indicating continuity of movement of the subject 2 and an image indicating continuity of answers of the subject 2 may be displayed or printed separately from each other.

Also, in the above-explained embodiment, timings at which the subject 2 made answers are measured by the user hearing the reproduced voice. However, the processing section 31 may measure timings of the answers by voice recognition processing. Alternatively, the processing section 31 may generate data indicating volume in time series and cause the display section 35 to display an image corresponding to the generated data. In the above configuration, the user can recognize timings of the answers based on the image (peak values) displayed on the display section 35. Further, in the above configuration, the user may evaluate continuity of answers (whether or not the subject 2 continued answering for the specific period of time) based on the image (peak values) displayed on the display section 35.

Also, in the above-explained embodiment, the user evaluates continuity of answers of the subject 2 by viewing an image indicating timings at which the subject 2 made answers. However, continuity of answers may be determined based on the reproduced voice only, without generating an image indicating timings at which the subject 2 made answers.

Also, in the above-explained embodiment, the user evaluates continuity of movement by viewing an image indicating timings at which the subject 2 put down his feet on the ground. However, the processing section 31 may determine continuity of movement (whether or not motion of the subject 2 continued for the specific period of time) based on the motion capture data. That is, the processing section 31 may function as an analyzing section.

Also, in the above-explained embodiment, the dual-task performing ability evaluation system 1c includes the motion capture device as the motion detecting section 5. However, as explained in the fourth embodiment, the dual-task performing ability evaluation system 1c may include as the motion detecting section 5, for example a vibration detecting sensor, an imaging device, or a mat-like pressure sensitive switch in order to determine continuity of movement of the subject 2.

For example, in a configuration in which a vibration detecting sensor is used as the motion detecting section 5, the motion detecting section 5 generates signals indicating timings at which vibration was generated by movement of the subject 2. Also, in a configuration in which a mat-like pressure sensitive switch is used as the motion detecting section 5, the motion detecting section 5 generates signals indicating timings at which the subject 2 stepped on a mat. The above signals indicate timings at which the subject 2 put down his feet on the ground. In a configuration in which the dual-task performing ability evaluation system 1c includes the vibration detecting sensor or the mat-like pressure sensitive switch, the processing section 31 can function as an analyzing section and determine continuity of movement of the subject 2.

Also, in the above-explained embodiment, data indicating continuity of movement of the subject 2 indicates timings at which the subject 2 put down his feet on the ground. However, data indicating continuity of movement of the subject 2 is not limited to the above data. For example, the processing section 31 may generate human body skeleton model data as data indicating continuity of movement of the subject 2. The human body skeleton model data can be generated based on motion capture data. In the above configuration, the processing section 31 may determine continuity of movement of the subject 2 based on the human body skeleton model data. That is, the processing section 31 may function as an analyzing section.

Also, in the fifth embodiment, the dual-task performing ability evaluation system 1c includes the microphone as the answer detecting section 4. However, the dual-task performing ability evaluation system 1c is not limited to the above configuration. When the intelligence task requires the subject to answer by motion, the dual-task performing ability evaluation system 1c can detect answers of the subject 2 using the motion detecting section 5.

Specifically, the processing section 31 can generate for example human body skeleton model data as data indicating continuity of movement and answers of the subject 2. Accordingly, the dual-task performing ability evaluation system 1c can display an image of a human body skeleton model for example on the display section 35. Through the above, a user of the dual-task performing ability evaluation system 1c can check motion of the subject 2 (the human body skeleton model data) and evaluate continuity of movement and answers of the subject 2.

In the configuration in which the human body skeleton model data is generated as data indicating continuity of movement and answers of the subject 2, the processing section 31 may function as an analyzing section and analyze the human body skeleton model data. That is, the processing section 31 may determine continuity of movement and answers of the subject 2 based on the human body skeleton model data.

Also, when the intelligence task requires the subject to answer by motion, an imaging device including a microphone may for example be used as the answer detecting section 4 and the motion detecting section 5.

Also, when the intelligence task is a task of changing a viewing direction according to instructions of an examiner, the dual-task performing ability evaluation system 1c includes, as the answer detecting section, a viewing direction detecting section that detects a viewing direction of the subject 2. In the above configuration, the processing section 31 generates viewing direction data as data indicating continuity of answers of the subject 2. Through the above, the dual-task performing ability evaluation system 1c can cause for example the display section 35 to display an image indicating a viewing direction of the subject 2. Consequently, a user of the dual-task performing ability evaluation system 1c can check the viewing direction of the subject 2 (viewing direction data) and evaluate continuity of answers of the subject 2.

In the configuration in which the dual-task performing ability evaluation system 1c includes the viewing direction detecting section, the processing section 31 may function as an analyzing section and analyze the viewing direction data. That is, the processing section 31 may determine continuity of answers of the subject 2 based on the viewing direction data.

Also, when the intelligence task is a task of pressing a switch according to instructions of an examiner, the dual-task performing ability evaluation system 1c includes a switch as the answer detecting section 4. In the above configuration, the answer detecting section 4 generates signals indicating whether or not the subject 2 pressed the switch. The signals indicate continuity of answers of the subject 2.

Based on output of the answer detecting section 4, the processing section 31 generates for example data indicating timings at which the subject 2 pressed the switch in time series. Through the above, the dual-task performing ability evaluation system 1c can display on the display section 35 an image indicating the timings at which the subject 2 pressed the switch in time series. Consequently, a user of the dual-task performing ability evaluation system 1c can check the timings at which the subject 2 pressed the switch (signals indicating whether or not the switch was pressed) and evaluate continuity of answers of the subject 2.

In the configuration in which the dual-task performing ability evaluation system 1a includes a switch, the processing section 31 may function as an analyzing section. That is, the processing section 31 may analyze signals indicating whether or not the subject 2 pressed the switch and determine continuity of answers of the subject 2.

Also, when the intelligence task is a task of pressing two switches according to instructions of an examiner, the dual-task performing ability evaluation system 1c includes two switches as the answer detecting section 4. In the above configuration, the answer detecting section 4 generates signals indicating which of the switches the subject 2 pressed. The signals indicate continuity of answers of the subject 2.

Based on output of the answer detecting section 4, the processing section 31 generates for example data indicating which of the switches the subject 2 pressed in time series. Through the above, the dual-task performing ability evaluation system 1c can display on the display section 35 an image indicating which of the switches the subject 2 pressed in time series. Consequently, a user of the dual-task performing ability evaluation system 1c can check which of the switches the subject 2 pressed (signals indicating which of the switches was pressed) and evaluate continuity of answers of the subject 2.

In the configuration in which the dual-task performing ability evaluation system 1a includes two switches, the processing section 31 may function as an analyzing section. That is, the processing section 31 may analyze signals indicating which of the switches the subject 2 pressed and determine continuity of answers of the subject 2.

### Sixth Embodiment

Next, a dual-task performing ability evaluation method according to a sixth embodiment, the dual-task performing ability evaluation system 1a, and a method for evaluating dual-task performing ability by using the dual-task performing ability evaluation system 1a will be explained with reference to FIGS. 2 and 13. Matter different from the first through fifth embodiments will be explained and explanation of the same matter as the first through fifth embodiments will be omitted. The sixth embodiment differs from the first through fifth embodiments in that dual-task performing ability is evaluated based on variation of intervals between answers of a subject performing a dual task.

FIG. 13 is a diagram illustrating a flow of the dual-task performing ability evaluation method according to the sixth embodiment. As illustrated in FIG. 13, the dual-task performing ability evaluation method according to the sixth embodiment includes a dual task step S206, an analysis step S406, and an evaluation step S606.

In the dual task step S206, a subject performs a dual task for a specific period of time. Further, in the dual task step S206, answers of the subject performing the dual task are detected.

In the analysis step S406, the answers of the subject to an intelligence task are analyzed. Specifically, in the sixth embodiment, variation of intervals between the answers of the subject is analyzed. For example, a standard deviation of the intervals between the answers can be calculated as the variation of the intervals between the answers.

In the evaluation step S606, dual-task performing ability of the subject is evaluated based on a result of the analysis obtained in the analysis step S406. Specifically, in the sixth embodiment, the dual-task performing ability of the subject is evaluated based on the variation of the intervals between the answers obtained in the analysis step S406.

The dual-task performing ability evaluated in the evaluation step S606 corresponds to a commonly used intelligence evaluation scale such as an MMSE score or a Hasegawa's Dementia Scale. In the sixth embodiment, it is determined whether or not an intelligence evaluation scale of the subject is no greater than a specific value. The specific value may change according to difficulty levels of a movement task and an intelligence task.

For example, when straight walking for one minute is employed as the movement task and serial subtraction of successively subtracting 1 from a preceding value by starting from 100 is employed as the intelligence task, it is determined whether or not an MMSE score of the subject is no greater than 23. Specifically, when a value of the standard deviation of the intervals between the answers of the subject is greater than a specific value (e.g., one second), the MMSE score of the subject is determined to be no greater than 23.

Next, the dual-task performing ability evaluation system 1a according to the sixth embodiment will be explained with reference to FIG. 2. The dual-task performing ability evaluation method illustrated in FIG. 13 is performed using the dual-task performing ability evaluation system 1a.

In the sixth embodiment, the answer detecting section 4 is a microphone. Also, the processing section 31 calculates a standard deviation of intervals between answers based on data indicating timings at which the subject made the answers. That is, the processing section 31 functions as an analyzing section that analyzes answers of the subject 2. Also, the processing section 31 generates an image that indicates a value of the standard deviation of the intervals between the answers.

Next, the method for evaluating dual-task performing ability of the subject 2 by using the dual-task performing ability evaluation system 1a will be explained. Specifically, a method for determining an intelligence evaluation scale of the subject 2 will be explained.

In determining the intelligence evaluation scale of the subject 2, a user initially stores (records) in the storage section 32 audio data corresponding to voice uttered by the subject 2 performing a dual task for a specific period of time.

Next, the user operates the input section 34 to cause the audio output section 33 to output sound corresponding to the audio data stored in the storage section 32. That is, the voice uttered by the subject 2 while performing the dual task is reproduced. Further, the user causes the display section 35 to display an elapsed time from initiation of the reproduction. The user measures timings (elapsed time from initiation of the reproduction) at which the subject 2 made answers based on the reproduced voice and the displayed elapsed time.

Next, the user operates the input section 34 to input data indicating timings of the answers. In response, the processing section 31 calculates a standard deviation of intervals between the answers and generates data indicating a value of the standard deviation of the intervals between the answers. Then, the processing section 31 causes the display section 35 to display an image indicating the value of the standard deviation of the intervals between the answers.

The user determines the intelligence evaluation scale of the subject 2 based on the value of the standard deviation of the intervals between the answers, which is displayed on the display section 35 (screen 35a). For example, when straight walking for one minute is employed as the movement task and serial subtraction of successively subtracting 1 from a preceding value by starting from 100 is employed as the intelligence task, it can be determined whether or not the MMSE score of the subject 2 is no greater than 23.

As described above, according to the sixth embodiment, dual-task performing ability corresponding to an intelligence evaluation scale can be evaluated by having a subject perform a dual task. Therefore, a degree of dementia can be evaluated more easily in a shorter time as compared with a case where dementia is diagnosed through a medical interview such as an MMSE or an intelligence test.

Also, according to the sixth embodiment, the system can be simplified similarly to the first embodiment as compared with a case where a mental disorder such as dementia is diagnosed by using nerve image processing technologies.

Also, similarly to the first embodiment, the subject can individually conduct diagnosis of a mental disorder such as dementia by using the dual-task performing ability evaluation system 1a.

Similarly to the first embodiment, devices such as an IC recorder and a smartphone may be used as an element that implements a function of recording audio data.

Also, the image indicating the value of the standard deviation is displayed on the display section 35 (data output section) included in the information processing device 3. However, the image may be displayed for example on a display (data output section) that is connected as an external device to the information processing device 3. Also, the image indicating the value of the standard deviation may for example be printed on a recording medium by a printer (data output section) that is connected as an external device to the information processing device 3.

Also, in the above-explained embodiment, timings at which the subject 2 made answers are measured by the user hearing the reproduced voice. However, the processing section 31 may measure timings of the answers by voice recognition processing. Alternatively, the processing section 31 may generate data indicating volume in time series and cause the display section 35 to display an image corresponding to the generated data. In the above configuration, the user can recognize timings of the answers based on the image (peak values) displayed on the display section 35.

Also, in the above-explained embodiment, the user determines an intelligence evaluation scale such as the MMSE score of the subject 2 by checking the value of the standard deviation. However, the processing section 31 may determine an intelligence evaluation scale of the subject 2 by comparing the value of the standard deviation with a specific value (e.g., one second). That is, the processing section 31 may generate data indicating whether or not an intelligence evaluation scale of the subject 2 is no greater than a specific value, as evaluation data indicating the dual-task performing ability of the subject 2. That is, the processing section 31 can function as an evaluation data generating section that generates the evaluation data. In the above configuration, the processing section 31 generates an evaluation result image that indicates the intelligence evaluation scale of the subject 2, and causes the display section 35 to display the generated evaluation result image.

In the sixth embodiment, the intelligence task requires the subject to answer by utterance. However, as explained in the first embodiment, the intelligence task may require the subject to answer by motion. Alternatively, the intelligence task may be a task of changing a viewing direction according to instructions of an examiner, or a task of pressing a switch or switches according to instructions of an examiner.

Also, in the sixth embodiment explained above, the dual-task performing ability evaluation system 1a includes the microphone as the answer detecting section 4. However, the dual-task performing ability evaluation system 1a is not limited to the above configuration.

For example, when the intelligence task requires the subject to answer by motion, the dual-task performing ability evaluation system 1a includes, as the answer detecting section 4, a motion detecting section that detects motion of the subject 2. Specifically, the motion detecting section may for example be an imaging device or a motion capture device.

In a configuration in which the answer detecting section 4 (motion detecting section) is an imaging device, the dual-task performing ability evaluation system 1a causes for example the display section 35 to display an image of the subject 2 imaged by the imaging device, as explained in the first embodiment. Through the above, a user of the dual-task performing ability evaluation system 1a can check motion of the subject 2 and measure timings at which the subject 2 made answers.

Also, in a configuration in which the answer detecting section 4 (motion detecting section) is a motion capture device, the dual-task performing ability evaluation system 1a causes the display section 35 to display for example an image of a human body skeleton model, as explained in the first embodiment. Through the above, a user of the dual-task performing ability evaluation system 1a can check motion of the subject 2 (human body skeleton model data) and measure timings at which the subject 2 made answers. Alternatively, the processing section 31 may measure timings at which the subject 2 made answers based on motion capture data or the human body skeleton model data.

When the intelligence task is a task of changing a viewing direction according to instructions of an examiner, the dual-task performing ability evaluation system 1a includes, as the answer detecting section, a viewing direction detecting section that detects a viewing direction of the subject 2. In the above configuration, the dual-task performing ability evaluation system 1a generates viewing direction data and causes the display section 35 to display an image indicating a viewing direction of the subject 2, as explained in the first embodiment. Through the above, a user of the dual-task performing ability evaluation system 1a can check the viewing direction of the subject 2 (viewing direction data) and measure timings at which the subject 2 made answers. Alternatively, the processing section 31 may measure timings at which the subject 2 made answers based on the viewing direction data.

When the intelligence task is a task of pressing a switch or two switches according to instructions of an examiner, the dual-task performing ability evaluation system 1a includes a switch or two switches as the answer detecting section 4. In the above configuration, the answer detecting section 4 generates signals indicating whether or not the subject 2 pressed the switch(es). The processing section 31 generates data indicating timings at which the subject 2 pressed the switch(es) based on output of the answer detecting section 4. Further, the processing section 31 causes the display section 35 to display an image indicating the timings at which the subject 2 pressed the switch(es). Through the above, a user of the dual-task performing ability evaluation system 1a can check the timings at which the subject 2 pressed the switch(es) (signals indicating whether or not the switch(es) is/are pressed) and measure timings at which the subject 2 made answers. Alternatively, the processing section 31 may analyze the signals indicating whether or not the subject 2 pressed the switch(es) and measure timings at which the subject 2 made answers.

### Seventh Embodiment

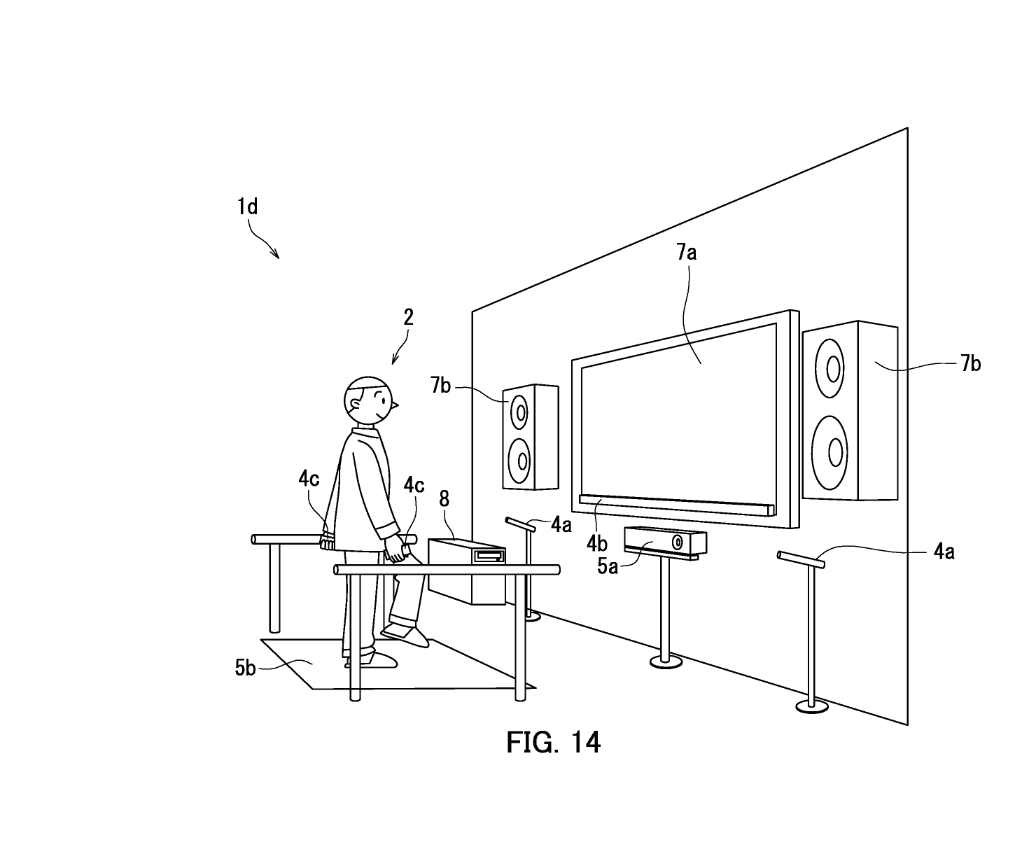

Next, a dual-task performing ability evaluation system 1d according to a seventh embodiment will be explained with reference to FIGS. 14 to 16. Matter different from the first through sixth embodiments will be explained, and explanation of the same matter as the first through sixth embodiments will be omitted. The seventh embodiment differs from the first through sixth embodiments in that the seventh embodiment includes a first task presenting section 7a that presents a task that the subject 2 should perform.

FIG. 14 is a diagram illustrating configuration of the dual-task performing ability evaluation system 1d. As illustrated in FIG. 14, the dual-task performing ability evaluation system 1d includes the first task presenting section 7a and a system controller 8. The first task presenting section 7a presents a task that the subject 2 should perform. In the seventh embodiment, the first task presenting section 7a is a display section such as a liquid-crystal display. The first task presenting section 7a displays (presents) a task that the subject 2 should perform, under control of the system controller 8. The system controller 8 may be for example a personal computer.

Also, the dual-task performing ability evaluation system 1d includes first answer detecting sections 4a, a second answer detecting section 4b, and third answer detecting sections 4c. The first through third answer detecting sections 4a to 4c detect an answer of the subject 2 to an intelligence task. The dual-task performing ability evaluation system 1d may include at least one of the first through third answer detecting sections 4a to 4c.

In the seventh embodiment, the first answer detecting sections 4a are directional microphones. The first answer detecting sections 4a (directional microphones) convert voice (an answer) uttered by the subject 2 performing an intelligence task to an electrical signal and send the electrical signal to the system controller 8.

In the seventh embodiment, the second answer detecting section 4b is a viewing direction detecting device. The second answer detecting section 4b includes for example a near infrared LED and an imaging device. The near infrared LED irradiates the eyes of the subject 2 with near infrared rays. The imaging device images the eyes of the subject 2. The system controller 8 analyzes an image or data captured by the imaging device and generates data indicating positions of pupils (a viewing direction) of the subject 2.

In the seventh embodiment, the third answer detecting sections 4c are answer switches. The subject 2 performs a task while holding the third answer detecting sections 4c in respective hands. The system controller 8 analyzes an answer of the subject 2 to an intelligence task for example based on which of the switches the subject 2 pressed.

The dual-task performing ability evaluation system 1d further includes a first motion detecting section 5a and a second motion detecting section 5b. The first motion detecting section 5a and the second motion detecting section 5b detect motion of the subject 2. In the seventh embodiment, the first motion detecting section 5a is a motion capture device and the second motion detecting section 5b is a mat-like pressure sensitive switch. The dual-task performing ability evaluation system 1d may include at least one of the first motion detecting section 5a and the second motion detecting section 5b.

The first motion detecting section 5a (motion capture device) generates motion capture data reflecting motion of parts of the subject 2. The system controller 8 generates data indicating a state of movement of the subject 2 based on the motion capture data. The dual-task performing ability evaluation system 1d (system controller 8) may generate data regarding an answer of the subject 2 to an intelligence task based on the motion capture data.

The second motion detecting section 5b (pressure sensitive switch) outputs signals corresponding to movement of feet of the subject 2. The system controller 8 generates data indicating a state of movement of the subject 2 based on the output of the second motion detecting section 5b. The dual-task performing ability evaluation system 1d (system controller 8) may generate data regarding an answer of the subject 2 to an intelligence task based on the output of the second motion detecting section 5b.

The dual-task performing ability evaluation system 1d may further include second task presenting sections 7b. In the seventh embodiment, the second task presenting sections 7b are speakers. The dual-task performing ability evaluation system 1d can output sound that notifies a task that the subject 2 should perform, by using the second task presenting sections 7b.

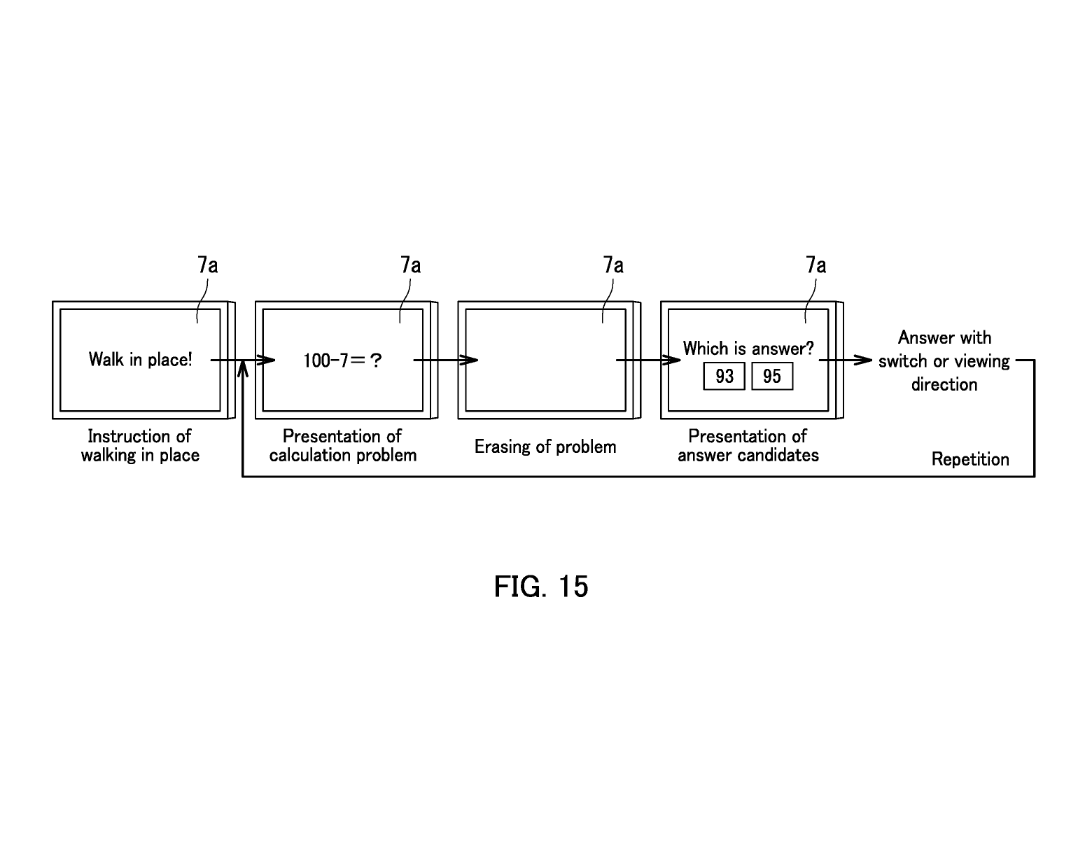

FIG. 15 is a diagram illustrating an example of a task that the dual-task performing ability evaluation system 1d presents to the subject 2. Specifically, FIG. 15 illustrates an example of an intelligence task that the first task presenting section 7a (display section) presents (displays) when a dual task is imposed on the subject 2 subsequently to a movement task (single task). In the example illustrated in FIG. 15, a calculation problem is presented as the intelligence task.

As illustrated in FIG. 15, the first task presenting section 7a initially presents a movement task (walking in place) that the subject 2 should perform. That is, a movement task that the subject 2 should perform is displayed on the first task presenting section 7a (display section). Then, after a specific period of time (period of time for performing the single task) has elapsed, the first task presenting section 7a presents a problem (calculation problem) that the subject 2 should answer. That is, an intelligence task (problem) that the subject 2 should perform is displayed on the first task presenting section 7a (display section). The first task presenting section 7a terminates presentation of the problem at a specific timing. That is, the problem is erased from the first task presenting section 7a (display section).

Thereafter, the first task presenting section 7a presents plural (two in FIG. 15) answer candidates. That is, plural answer candidates are displayed on the first task presenting section 7a (display section). The subject 2 chooses an answer by using the third answer detecting sections 4c (answer switches) held in the respective hands. Alternatively, the subject 2 chooses an answer by a viewing direction.

Once the subject 2 chooses an answer, the first task presenting section 7a presents as the next problem, a problem different from that the subject 2 presently answered. Afterwards, presentation of a problem that the subject 2 should answer is repeated until a specific period of time for performing the dual task elapses.

The system controller 8 analyzes (calculates) for example at least one of a total number of answers, the number of right answers, a right answer ratio, an average interval between answers, and a standard deviation of intervals between answers, based on output of the third answer detecting sections 4c or the second answer detecting section 4b.

Meanwhile, the first motion detecting section 5a (motion capture device) generates motion capture data reflecting motion of parts of the subject 2 performing the movement task (single task) and the dual task. Alternatively, the system controller 8 generates human body skeleton model data based on motion capture data.

Also, the second motion detecting section 5b (pressure sensitive switch) generates signals indicating timings at which the subject 2 stepped on a mat while performing the movement task (single task) and the dual task. The system controller 8 analyzes motion of the subject 2 based on output of the second motion detecting section 5b.

For example, the system controller 8 generates data indicating a stepping cycle (stepping rate) of the subject 2 as explained in the second embodiment, based on output of the first motion detecting section 5a or output of the second motion detecting section 5b.

According to the seventh embodiment explained above, an answer of the subject 2 can be determined as right or wrong in real time. Accordingly, a difficulty level of the intelligence task imposed on the subject 2 can be adjusted in real time. For example, the difficulty level of the intelligence task can be adjusted by increasing or decreasing the number of answer candidates. Alternatively, a difficulty level of the calculation problem per se may be adjusted.

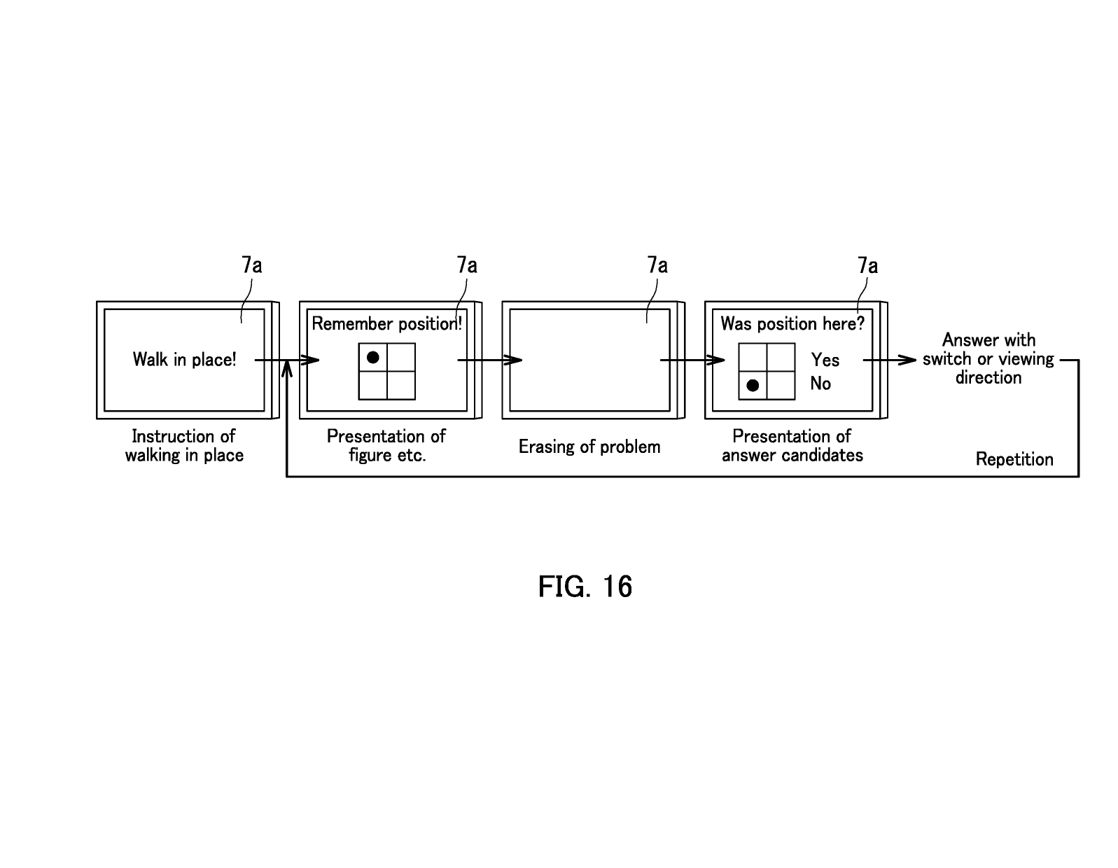

The intelligence task that the first task presenting section 7a presents is not limited to a calculation problem. For example, the intelligence task may be a problem that successively presents numbers and makes the subject answer the n-th number as counted from the lastly presented number. Also, the intelligence task may be a position memory problem as illustrated in FIG. 16.

FIG. 16 is a diagram illustrating another example of a task that the dual-task performing ability evaluation system 1d presents to the subject 2. Specifically, FIG. 16 illustrates another example of the intelligence task that the first task presenting section 7a (display section) presents (displays) when a dual task is imposed on the subject 2 subsequently to a movement task (single task).

As illustrated in FIG. 16, the first task presenting section 7a initially presents a movement task (walking in place) that the subject 2 should perform. Then, after a specific period of time (period of time for performing the single task) has elapsed, the first task presenting section 7a presents a problem (position memory problem) that the subject 2 should answer. The first task presenting section 7a terminates presentation of the problem at a specific timing.

Thereafter, the first task presenting section 7a presents plural answer candidates (two candidates of “Yes” and “No” in FIG. 16). The subject 2 chooses an answer by using the third answer detecting sections 4c (answer switches) held in the respective hands. Alternatively, the subject 2 chooses an answer by a viewing direction.

Once the subject 2 chooses an answer, the first task presenting section 7a presents as the next problem, a problem in which a figure is placed at a position different from that presently answered. Afterwards, presentation of a problem that the subject 2 should answer is repeated until a specific period of time for performing the dual task elapses.

In the case of the position memory problem explained with reference to FIG. 16, a difficulty level of the intelligence task can be adjusted for example by increasing or decreasing the number of positions at which a figure can be placed. That is, FIG. 16 illustrates a case where a figure can be placed at “four” positions. When presenting an intelligence task that is more difficult than that illustrated in FIG. 16, the number of positions at which a figure can be placed is set to be five or more.

Alternatively, the difficulty level of the intelligence task may be adjusted by increasing or decreasing the number of figures. FIG. 16 illustrates a case where the number of figures is “one”. That is, in the intelligence task illustrated in FIG. 16, the number of positions that should be remembered at a time is “one”. Accordingly, when presenting an intelligence task that is more difficult than that illustrated in FIG. 16, the number of figures (the number of positions of figures that should be remembered at a time) is set to be two or more. The number of positions at which the figures can be placed is also increased along with an increase of the number of figures.

Also, in the above-explained embodiment, the dual-task performing ability evaluation system 1d includes the first answer detecting sections 4a, the second answer detecting section 4b, the third answer detecting sections 4c, the first motion detecting section 5a, the second motion detecting section 5b, the first task presenting section 7a, the second task presenting sections 7b, and the system controller 8. However, the dual-task performing ability evaluation system 1d is not limited to the above configuration. For example, the dual-task performing ability evaluation system 1d may include one of the first answer detecting sections 4a, the second answer detecting section 4b, the third answer detecting sections 4c, the first motion detecting section 5a, and the second motion detecting section 5b. Also, the dual-task performing ability evaluation system 1d may include one of the first task presenting section 7a and the second task presenting sections 7b.

Also, the dual-task performing ability evaluation system 1d may be a single device that can process information such as a notebook personal computer, a smartphone, or a tablet terminal. For example, in a configuration in which the dual-task performing ability evaluation system 1d is a smartphone, the dual-task performing ability evaluation system 1d includes the first task presenting section 7a, the third answer detecting sections 4c, and the system controller 8. Specifically, a display section of the smartphone functions as the first task presenting section 7a. Also, a touch panel sensor of the smartphone functions as the third answer detecting sections 4c. That is, the touch panel sensor functions as an answer switch. Also, a processing section of the smartphone functions as the system controller 8.

### Eighth Embodiment

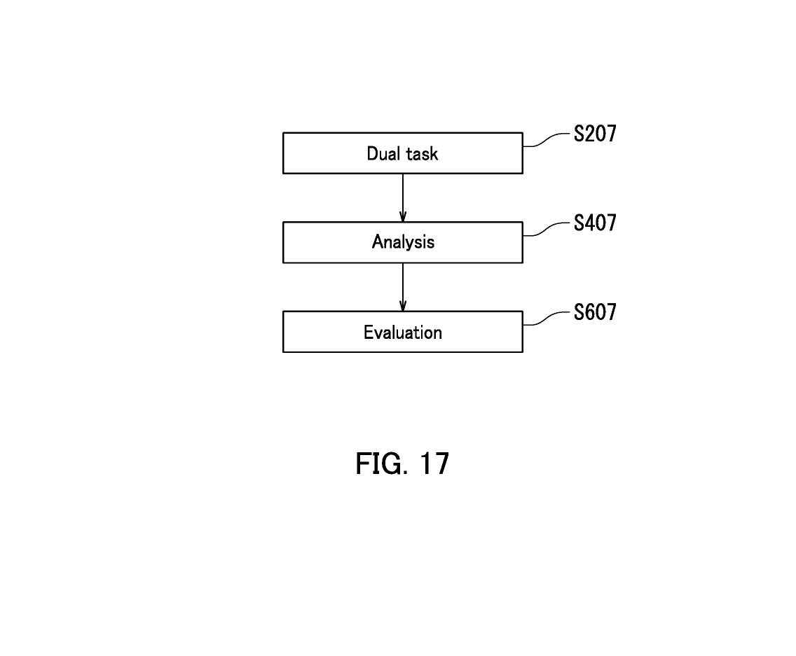

Next, a dual-task performing ability evaluation system 1e according to an eighth embodiment will be explained with reference to FIGS. 17 to 19. Matter different from the first through seventh embodiments will be explained and explanation of the same matter as the first through seventh embodiments will be omitted. The eighth embodiment differs from the first through seventh embodiments in contents of evaluation data (an evaluation result) generated in an evaluation step.

Initially, a dual-task performing ability evaluation method according to the eighth embodiment will be explained with reference to FIG. 17. FIG. 17 is a diagram illustrating a flow of the dual-task performing ability evaluation method according to the eighth embodiment. As illustrated in FIG. 17, the dual-task performing ability evaluation method according to the eighth embodiment includes a dual task step S207, an analysis step S407, and an evaluation step S607.

In the dual task step S207, a subject performs a dual task for a specific period of time. Further, in the dual task step S207, at least one of motion and answers of the subject performing the dual task is detected.

In the analysis step S407, at least one of the motion and the answers of the subject while performing the dual task step S207 is analyzed. For example, when a movement task included in the dual task is a task that makes the subject walk in place, an average value of one-step times or a standard deviation of one-step times is measured as a characteristic amount of the motion of the subject. Also, a right answer ratio or an average value of intervals between the answers is measured as a characteristic amount of the answers of the subject.

In the evaluation step S607, dual-task performing ability of the subject is evaluated based on a result of the analysis (the characteristic amount of the motion of the subject or the characteristic amount of the answers of the subject) obtained in the analysis step S407. Further, in the evaluation step S607, evaluation data indicating the dual-task performing ability of the subject is generated.

Specifically, in the eighth embodiment, the result of the analysis is compared with a standard value for each age based on the actual age of the subject. Based on a result of the comparison, current cognitive ability of the subject or a current health degree of the subject's brain is determined. Alternatively, a current brain age of the subject is determined by comparing the result of the analysis with a standard value for each age. When the actual age of the subject is 15 years old or younger, the determined cognitive ability, health degree of the brain, or brain age indicates a degree of intellectual growth such as a learning level.

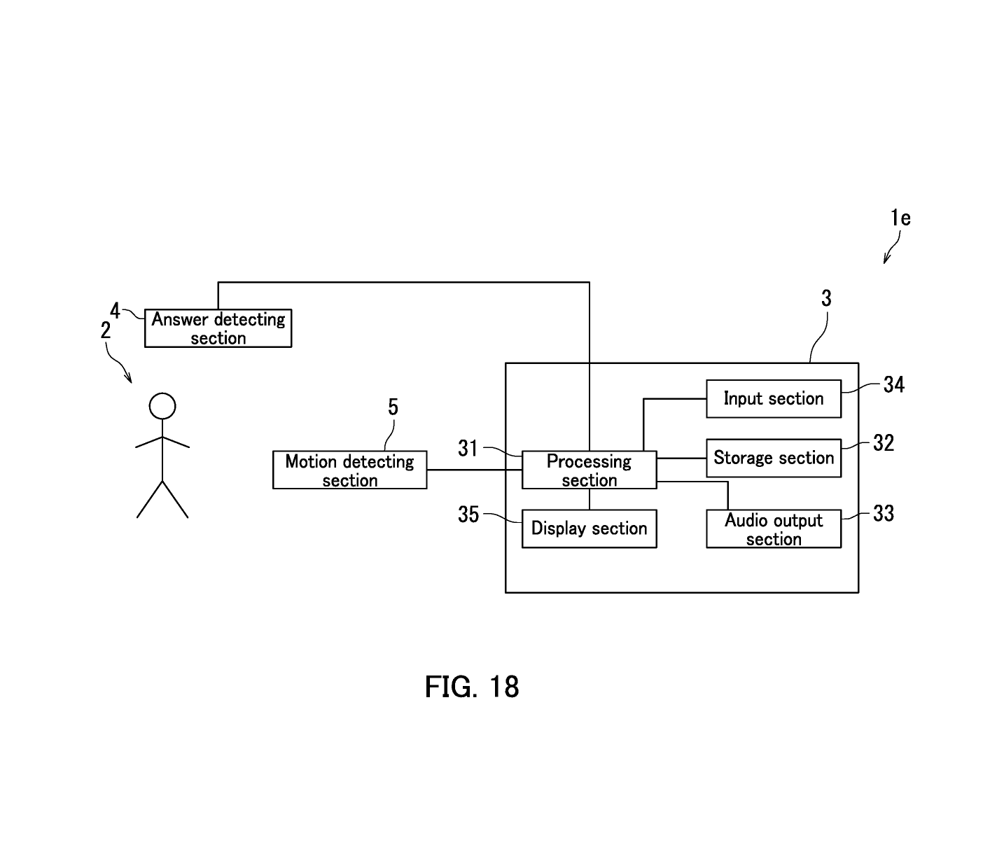

Next, the dual-task performing ability evaluation system 1e according to the eighth embodiment will be explained with reference to FIG. 18. FIG. 18 is a block diagram illustrating configuration of the dual-task performing ability evaluation system 1e. The dual-task performing ability evaluation method illustrated in FIG. 17 is performed using the dual-task performing ability evaluation system 1e. As illustrated in FIG. 18, the dual-task performing ability evaluation system 1e includes the information processing device 3, the answer detecting section 4, and the motion detecting section 5.

The answer detecting section 4 detects an answer of the subject 2 performing a dual task. The answer detecting section 4 may include for example at least one of a microphone, a viewing direction detecting device, and an answer switch.

The motion detecting section 5 detects motion of the subject 2 performing the dual task. The motion detecting section 5 may include for example at least one of a motion capture device, an imaging device, a vibration detecting sensor, and a mat-like pressure sensitive switch. When an intelligence task requires the subject 2 to answer by motion, the motion detecting section 5 can also function as the answer detecting section 4. In the above configuration, the answer detecting section 4 may be omitted.

In the eighth embodiment, the storage section 32 stores therein standard value data of characteristic amounts of motion and answers corresponding to a specific dual task. Hereinafter, standard value data of a characteristic amount of motion may be referred to as standard value data of motion, and standard value data of a characteristic amount of answers may be referred to as standard value data of answers. Similarly, a standard value of a characteristic amount of motion may be referred to as a standard value of motion, and a standard value of a characteristic amount of answers may be referred to as a standard value of answers. For example, the storage section 32 can store therein an average value of one-step times for each age as standard value data of motion corresponding to a dual task that requires the subject 2 to perform “walking in place” as a movement task and “serial subtraction” as an intelligence task. Further, the storage section 32 can store therein an average interval between answers for each age as standard value data of answers.

Data indicating a characteristic amount of motion or a characteristic amount of answers of the subject 2 performing the dual task (data indicating an analysis result) is input to the processing section 31 through the input section 34. For example, in a configuration in which the answer detecting section 4 detects voice (answers) uttered by the subject 2 and the processing section 31 reproduces the voice (sound) uttered by the subject 2 using the audio output section 33, a user can measure timings at which the subject 2 made the answers based on the reproduced voice. The user calculates for example an average value of intervals between the answers of the subject 2 based on a result of the measurement and inputs data indicating the average value through the input section 34.

Alternatively, the processing section 31 may function as an analyzing section and measure a characteristic amount of motion or a characteristic amount of answers of the subject 2 performing the dual task. In the above configuration, the user need not input data indicating an analysis result, and the processing section 31 generates evaluation data based on data that the processing section 31 measured. For example, in a configuration in which the answer detecting section 4 detects voice (answers) uttered by the subject 2, the processing section 31 can measure timings of the answers by voice recognition processing and calculate for example an average value of intervals between the answers of the subject 2 based on a result of the measurement.

The processing section 31 generates evaluation data indicating the dual-task performing ability of the subject 2 based on the data indicating the characteristic amount of motion or the characteristic amount of answers of the subject 2 performing the dual task. That is, the processing section 31 functions as an evaluation data generating section.

Specifically, the processing section 31 calculates a difference between the characteristic amount of motion of the subject 2 and a standard value of motion for the actual age of the subject 2, based on the actual age of the subject 2. Alternatively, the processing section 31 calculates a difference between the characteristic amount of answers of the subject 2 and a standard value of answers for the actual age of the subject 2. Alternatively, the processing section 31 determines a brain age of the subject 2 based on the characteristic amount of motion or the characteristic amount of answers of the subject 2 by referring to the standard value data of motion or the standard value data of answers. Note that data indicating the actual age of the subject 2 is input through the input section 34 and stored in the storage section 32.

As described above, in the eighth embodiment, data indicating a difference between the characteristic amount of motion of the subject 2 and a standard value of motion for the actual age of the subject 2 is generated as evaluation data indicating the dual-task performing ability of the subject 2. Alternatively, data indicating a difference between the characteristic amount of answers of the subject 2 and a standard value of answers for the actual age of the subject 2 is generated as the evaluation data. Alternatively, data indicating a brain age of the subject 2 is generated as the evaluation data.

The processing section 31 generates an evaluation result image corresponding to the evaluation data and causes the display section 35 to display the generated evaluation result image. In a configuration in which the evaluation data indicates a difference between the characteristic amount of motion of the subject 2 and a standard value of motion for the actual age of the subject 2, the evaluation result image may be a specific image corresponding to the size of the difference. Similarly, in a configuration in which the evaluation data indicates a difference between the characteristic amount of answers of the subject 2 and a standard value of answers for the actual age of the subject 2, the evaluation result image may be a specific image corresponding to the size of the difference. The specific image may for example be an emoticon. The emoticon represents different expressions in accordance with a measured characteristic amount and the size of a difference between the characteristic amount and a standard value for the characteristic amount.

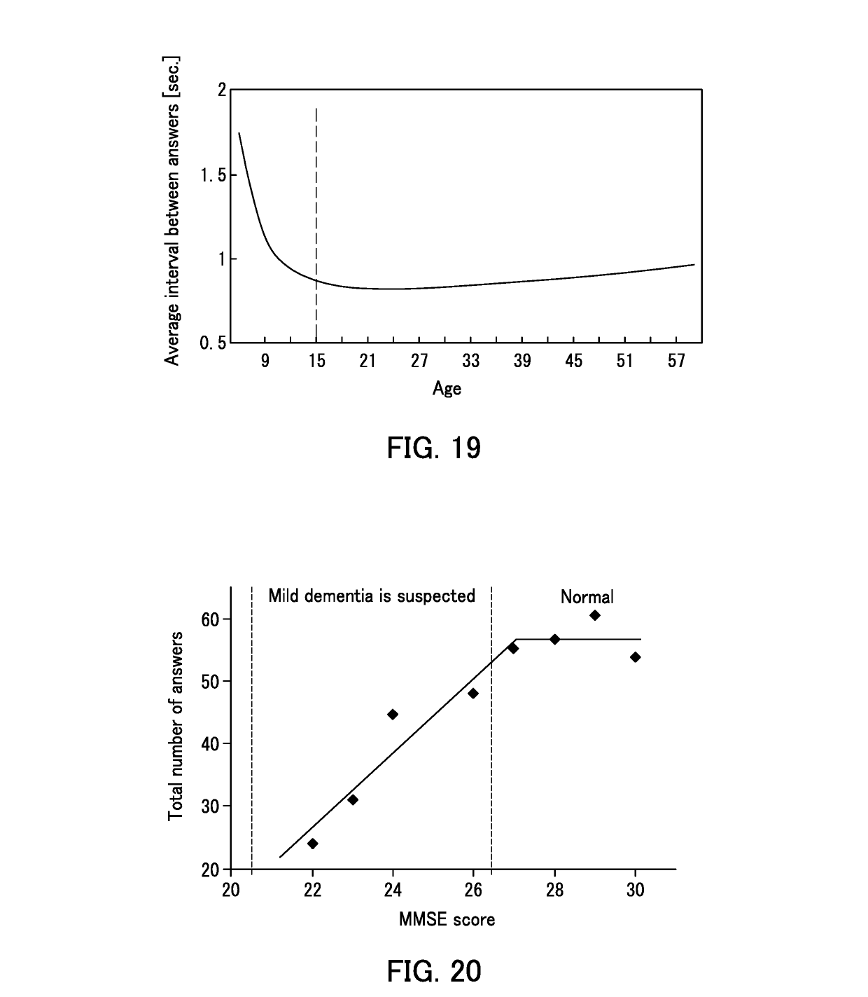

Next, standard value data will be explained with reference to FIG. 19. FIG. 19 is a diagram illustrating an example of standard value data. Specifically, the standard value data in FIG. 19 indicates a standard value of an average interval between answers for each age. In FIG. 19, the horizontal axis represents the age and the vertical axis represents the average interval between answers.

As illustrated in FIG. 19, the standard value of the average interval between answers decreases as the age increases from 6 years old to 15 years old. By contrast, the standard value of the average interval between answers barely changes above 15 years old.

According to the eighth embodiment, the dual-task performing ability of the subject 2 can be evaluated for example by comparing a measured average interval between answers of the subject 2 with a standard value of the average interval between answers indicated in FIG. 19. Specifically, current cognitive ability of the subject or a current health degree of the brain of the subject can be determined based on a difference between a measured average interval between answers of the subject 2 and a standard value for the actual age of the subject 2. Alternatively, a standard value corresponding to the measured average interval between answers of the subject 2 can be detected and an age corresponding to the detected standard value can be determined. The determined age indicates a current brain age of the subject 2.

In the above-explained embodiment, the dual-task performing ability evaluation system 1e includes the answer detecting section 4 and the motion detecting section 5. However, the dual-task performing ability evaluation system 1e may include either of the answer detecting section 4 and the motion detecting section 5. In the above configuration, the storage section 32 stores therein either of standard value data of a characteristic amount of motion and standard value data of a characteristic amount of answers.

Also, a single task step including a movement task only may be performed before or after the dual task step. In this case, motion of the subject is detected in the single task step. Also, the subject performs the same movement task in the single task step and the dual task step. For example, when the movement task is a task that makes the subject walk in place, a difference or ratio between an average value of one-step times of the subject while performing the single task and an average value of one-step times of the subject while performing the dual task can be calculated as a characteristic amount of motion of the subject. Alternatively, a difference or ratio between a standard deviation of one-step times of the subject while performing the single task and a standard deviation of one-step times of the subject while performing the dual task can be calculated as a characteristic amount of motion of the subject.

Through the above, the embodiments of the present invention have been explained with reference to the drawings. However, the present invention is not limited to the above-described embodiments and is practicable in various manners within the scope not departing from the gist of the present invention.

For example, in the embodiments of the present invention, front views of the human body skeleton models are displayed on the screen 35a. However, side views of the human body skeleton models may be displayed on the screen 35a. Alternatively, a front view of a human body skeleton model and a side view of the human body skeleton model may be simultaneously displayed on the screen 35a.

Also, matter explained in the embodiments of the present invention may be combined as appropriate. For example, it is possible to determine an MMSE score of a subject to be 27 or at least 28 by the dual-task performing ability evaluation method explained in the third embodiment after determining the MMSE score of the subject to be at least 27 by the dual-task performing ability evaluation method explained in the first embodiment. Also, for example, the dual-task performing ability evaluation systems 1a to 1e each may be a single device that can process information such as a notebook personal computer, a smartphone, or a tablet terminal as explained above in the seventh embodiment.

### EXAMPLES

Next, examples of the present invention will be explained. However, the present invention is not limited to the examples explained below.

### First Example

An MMSE was conducted on subjects to obtain an MMSE score of each subject. Thereafter, each subject performed a dual task of answering a problem of serial subtraction by successively subtracting 1 from a preceding value by starting from 100 while walking for one minute. The number of answers (including wrong answers) of each subject to the problem of serial subtraction was counted. Results are shown in FIG. 20.

FIG. 20 illustrates a relationship between the MMSE score and the total number of answers. The total number of answers of each subject is plotted for each MMSE score. In FIG. 20, the horizontal axis represents the MMSE score and the vertical axis represents the total number of answers.

As illustrated in FIG. 20, the total number of answers increases as the MMSE score increases within the range where the MMSE score is no greater than 27. Also, the present inventors obtained above-indicated Formula (4) by a linear regression method based on each plot point. FIG. 20 illustrates a graph of Formula (4). Note that the graph illustrated in FIG. 20 corresponds to MMSE scores smaller than 27.

### Second Example

The MMSE was conducted on subjects to obtain an MMSE score of each subject. Thereafter, each subject performed walking in place for one minute (a movement task). Subsequently, each subject performed a dual task of reciting expressions starting from a syllable “ka” while walking in place for one minute. It was determined for each subject whether or not a stepping rate was consistent between the movement task and the dual task. Also, it was determined for each subject whether or not a step width while walking in place was consistent between the movement task and the dual task. Results are shown in Table 1 below.

TABLE 1

| MMSE score | Stepping rate | Step width |
|---|---|---|
| 30-27 | 43 [%] | 73 [%] |
| 26-24 | 0 [%] | 70 [%] |
| 23-22 | 0 [%] | 25 [%] |

As shown in Table 1, within the range where the MMSE score is at least 27 and no greater than 30, a percentage of subjects whose stepping rate was consistent between the movement task and the dual task is 43%. By contrast, within the range where the MMSE score is at least 22 and no greater than 26, a percentage of subjects whose stepping rate was consistent between the movement task and the dual task is 0%. Consequently, the results show that when the stepping rate is consistent between the movement task and the dual task, the MMSE score is at least 27.

Also, within the range where the MMSE score is at least 24 and no greater than 30, a percentage of subjects whose step width while walking in place was consistent between the movement task and the dual task is at least 70%. By contrast, within the range where the MMSE score is at least 22 and no greater than 23, a percentage of subjects whose step width while walking in place was consistent between the movement task and the dual task is 25%. Consequently, the results show that when the step width while walking in place is consistent between the movement task and the dual task, the MMSE score is highly likely to be at least 24.

### Third Example

The MMSE was conducted on subjects to obtain an MMSE score of each subject. Thereafter, each subject performed walking in place for one minute (a movement task). Subsequently, each subject performed a first dual task of answering a problem of serial subtraction by successively subtracting 1 from a preceding value by starting from 100 while walking in place for one minute. Subsequently, each subject performed a second dual task of reciting expressions starting from a syllable “ka” while walking in place for one minute. Then, with respect to the following evaluation items: a height of a raised foot, a length of arm swing, a stepping rate, shortness of a step width, and slightness of wobbling of the upper half of the body, it was determined for each subject whether or not evaluation results of at least two of the evaluation items become lower in order of the movement task, the first dual task, and the second dual task. Results are shown in Table 2 below.

TABLE 2

| | Percentage of subjects with at least two items |
|---|---|
| MMSE score | satisfying specific conditions |
| 30-28 | 27 [%] |
| 27-26 | 80 [%] |
| 24-22 | 25 [%] |

As shown in Table 2, where the MMSE score is 26 or 27, a percentage of subjects for whom evaluation results of at least two of the evaluation items become lower in order of the movement task, the first dual task, and the second dual task is 80%. By contrast, within the range where the MMSE score is at least 22 and no greater than 24 and the range where the MMSE score is at least 28 and no greater than 30, a percentage of subjects for whom evaluation results of at least two of the evaluation items become lower in order of the movement task, the first dual task, and the second dual task is no greater than 27%. Consequently, the results show that when evaluation results of at least two of the evaluation items become lower in order of the movement task, the first dual task, and the second dual task, the MMSE score is highly likely to be 27 or 26.

### Fourth Example

The MMSE was conducted on subjects to obtain an MMSE score of each subject. Thereafter, each subject performed a first dual task of answering a problem of serial subtraction by successively subtracting 1 from a preceding value by starting from 100 while walking in place for one minute. Subsequently, each subject performed a second dual task of reciting expressions starting from a syllable “ka” while walking in place for one minute. It was determined for each subject whether or not the subject stopped walking in place. Results are shown in Table 3 below.

TABLE 3

| | Number of subjects stopped walking | |
|---|---|---|
| MMSE score | in place/Total number of subjects | |
| 25 or more | 0/22 | |
| 24 23 22 | 1/2 1/2 1/2 | 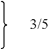 |

As shown in Table 3, no subjects stopped walking in place halfway within the range where the MMSE score is 25 or more. Consequently, the results show that when a subject stops walking in place halfway, the MMSE score of the subject is no greater than 24.

### Fifth Example

The MMSE was conducted on subjects to obtain an MMSE score of each subject. Thereafter, each subject performed a dual task of reciting expressions starting from a syllable “ka” while walking straight for one minute. It was determined for each subject whether or not the subject stopped walking and finished answering before one minute elapsed while performing the dual task.

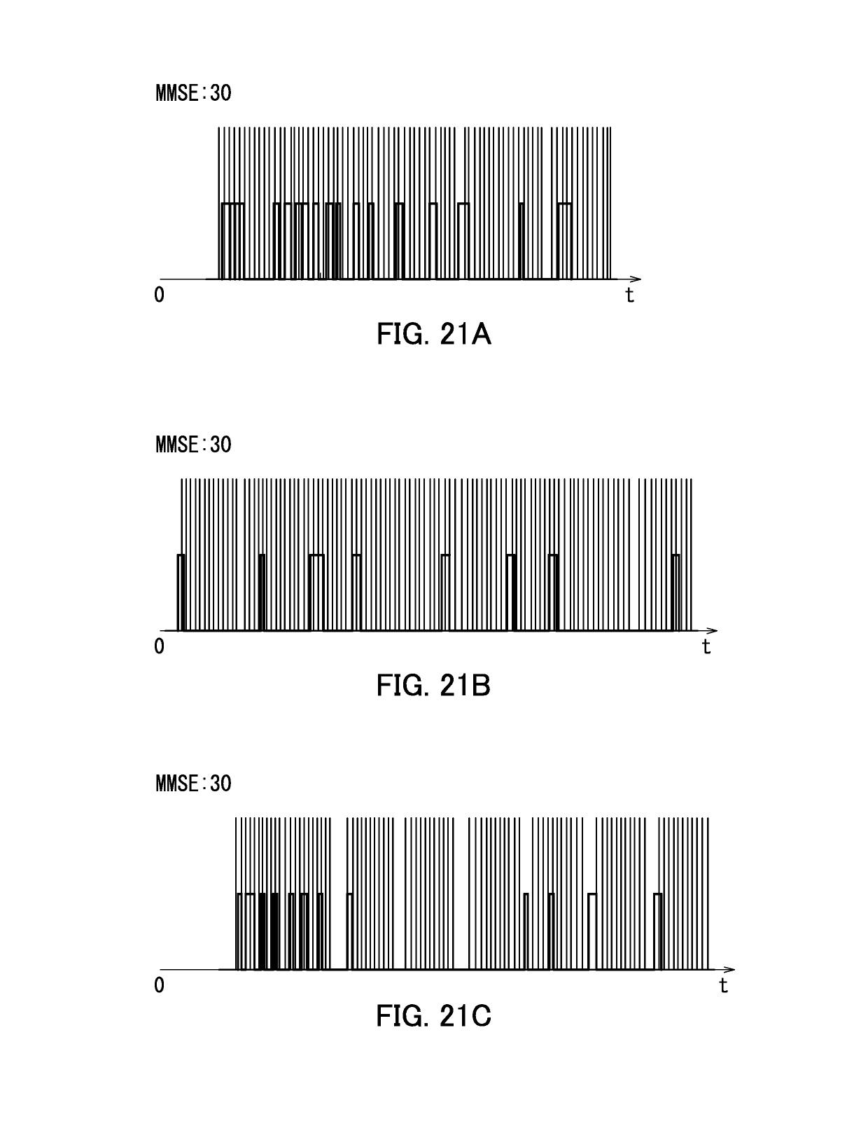

FIGS. 21A to 27 illustrate timings of motion and timings of answers of the subjects performing the dual task. Specifically, FIGS. 21A to 27 illustrate timings at which the subjects put down their feet on the ground and timings at which the subjects made answers. The horizontal axes in FIGS. 21A to 27 represent time. Also, in FIGS. 21A to 27, long thin lines indicate timings at which the subjects put down their feet on the ground and short bold lines indicate timings at which the subjects made answers to the problem of reciting expressions starting from a syllable “ka”.

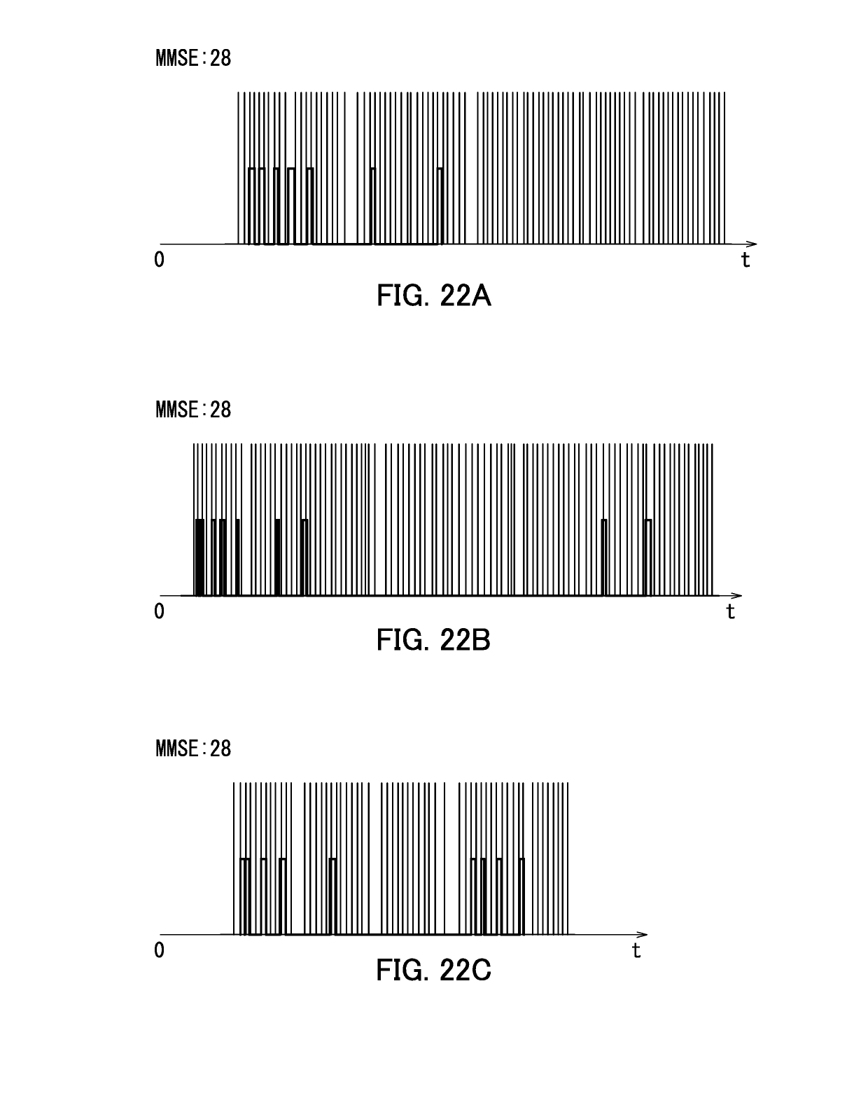

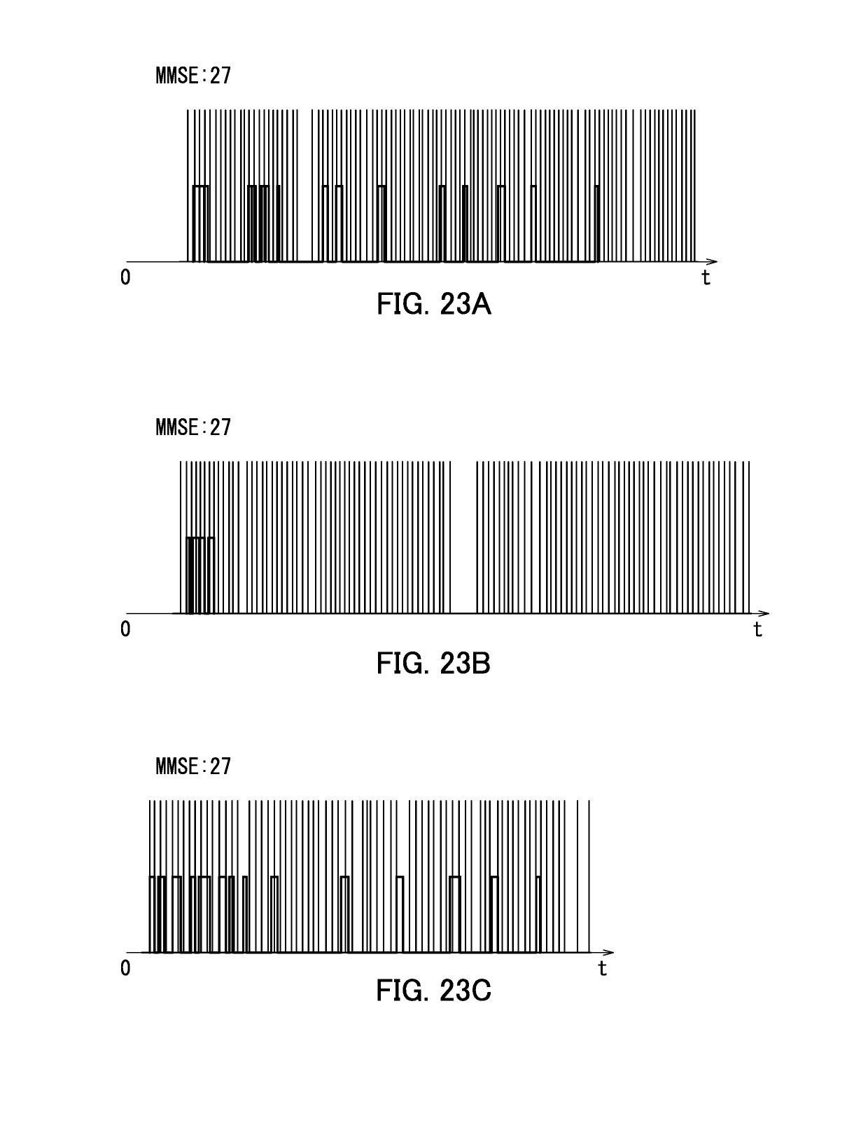

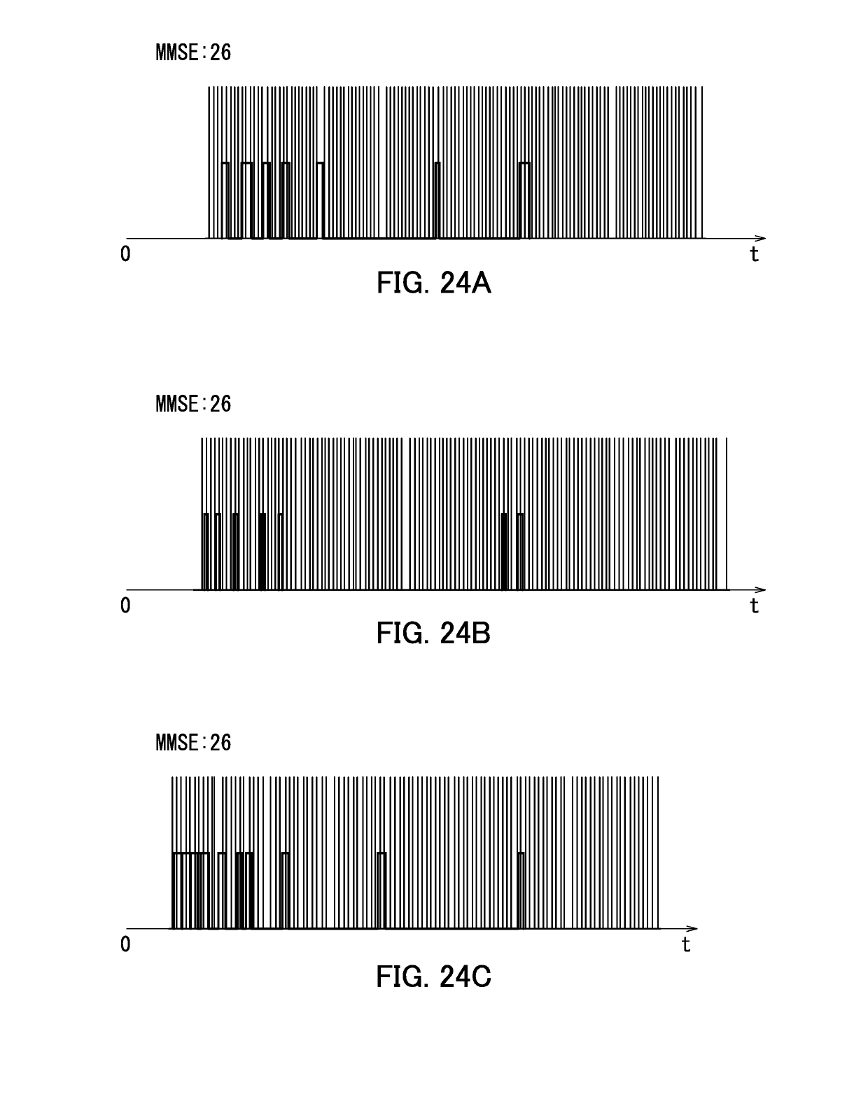

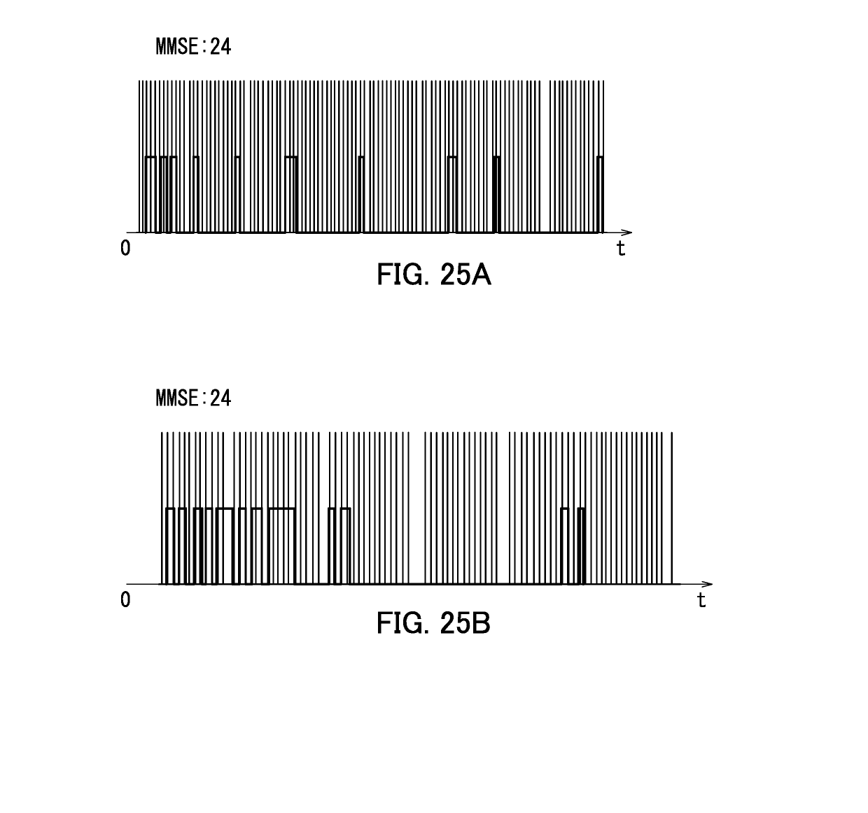

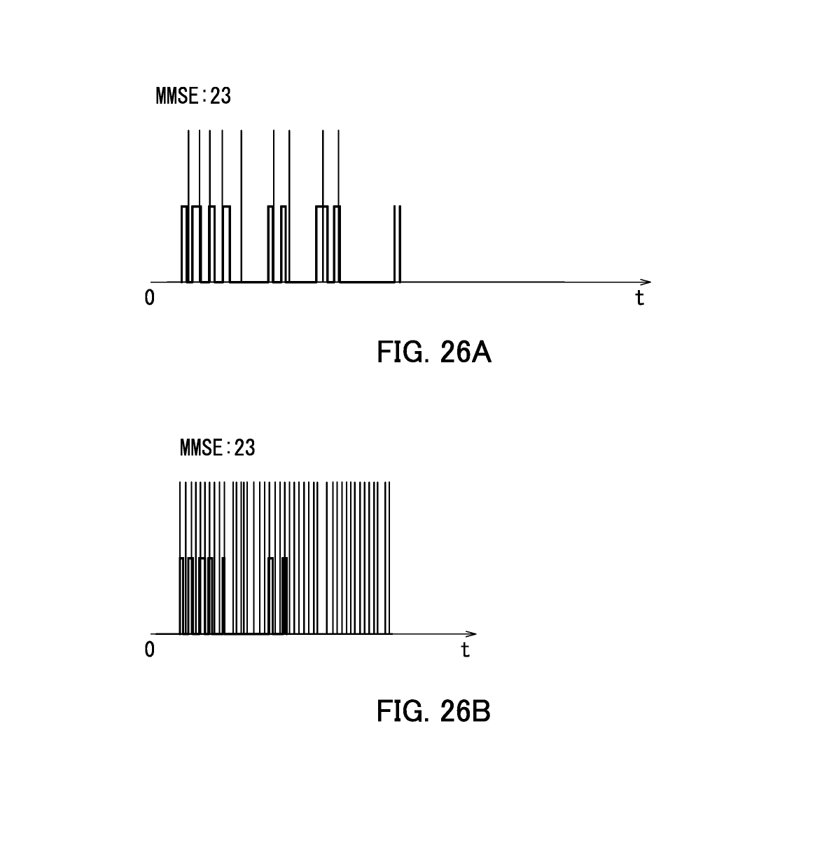

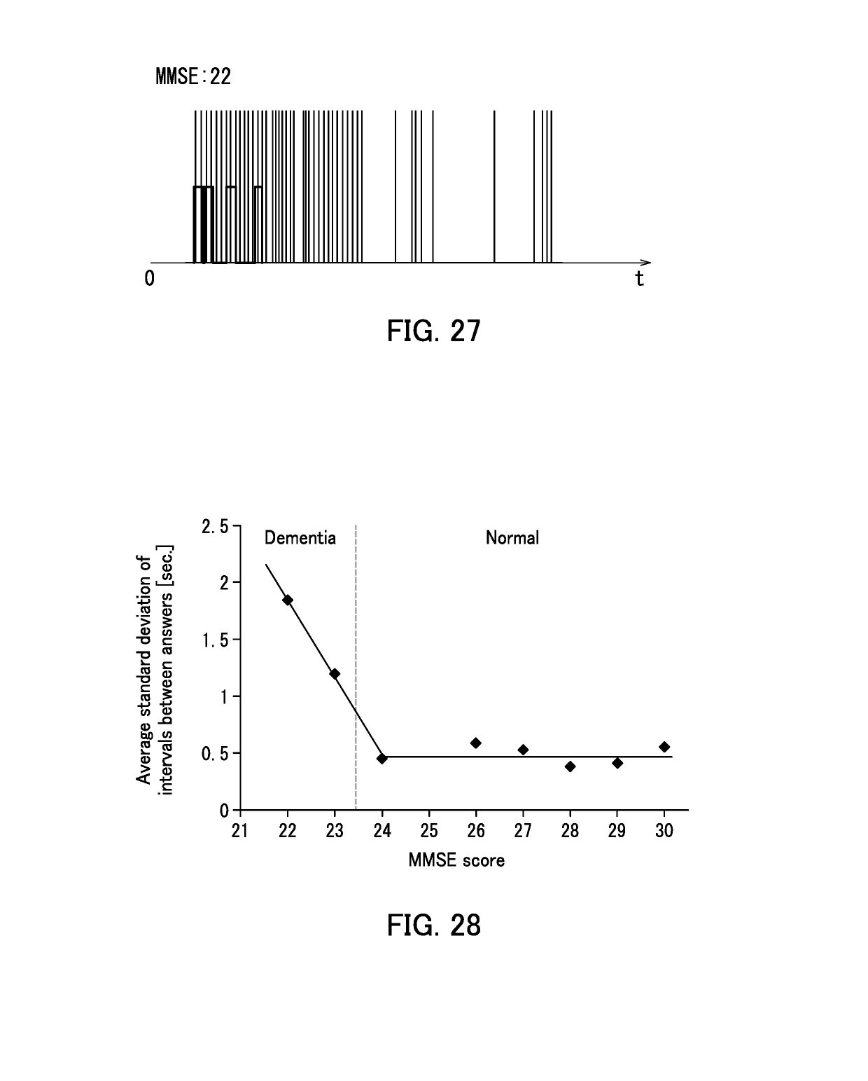

FIGS. 21A to 21C each illustrate timings of motion and timings of answers of a subject having an MMSE score of 30. FIGS. 22A to 22C each illustrate timings of motion and timings of answers of a subject having an MMSE score of 28. FIGS. 23A to 23C each illustrate timings of motion and timings of answers of a subject having an MMSE score of 27. FIGS. 24A to 24C each illustrate timings of motion and timings of answers of a subject having an MMSE score of 26. FIGS. 25A and 25B each illustrate timings of motion and timings of answers of a subject having an MMSE score of 24. FIGS. 26A and 26B each illustrate timings of motion and timings of answers of a subject having an MMSE score of 23. FIG. 27 illustrates timings of motion and timings of answers of a subject having an MMSE score of 22.

As illustrated in FIGS. 21A to 27, the results show that when a subject stops walking and finishes answering before one minute elapses while performing the dual task, the MMSE score of the subject is highly likely to be no greater than 23.

### Sixth Example

The MMSE was conducted on subjects to obtain an MMSE score of each subject. Thereafter, each subject performed a dual task of answering a problem of serial subtraction by successively subtracting 1 from a preceding value by starting from 100 while walking in place for one minute. Then, a standard deviation of intervals between answers of each subject to the problem of serial subtraction was calculated. Further, among subjects having the same MMSE score, an average value of the standard deviation of intervals between answers was calculated. Results are shown in FIG. 28.

FIG. 28 illustrates a relationship between the standard deviation of intervals between answers and the MMSE score. An average value of the standard deviation of intervals between answers is plotted for each MMSE score. In FIG. 28, the horizontal axis represents the MMSE score and the vertical axis represents the average value of the standard deviation of intervals between answers.

As illustrated in FIG. 28, the average value of the standard deviation of intervals between answers increases as the MMSE score decreases within the range where the MMSE score is no greater than 23. Also, the average value of the standard deviation of intervals between answers is one second or more within the range where the MMSE score is no greater than 23. Consequently, the results show that when a value of the standard deviation of intervals between answers is one second or more, the MMSE score is no greater than 23.

### Seventh Example

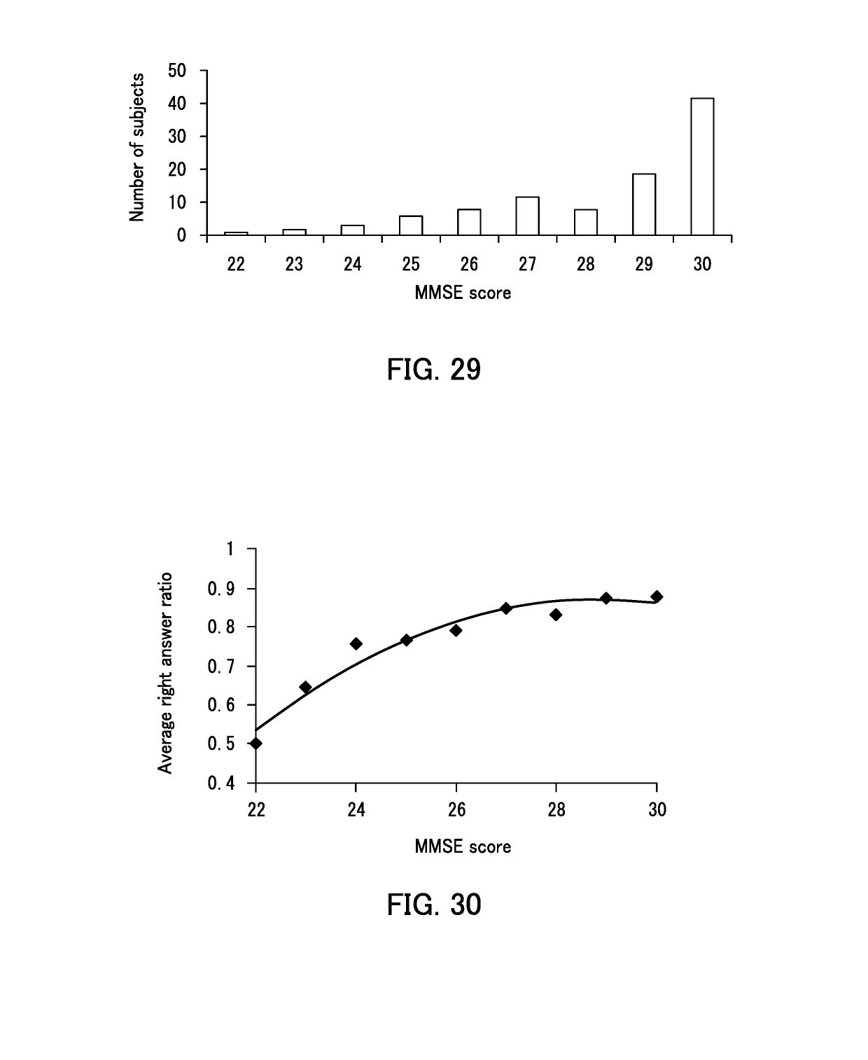

The MMSE was conducted on subjects to obtain an MMSE score of each subject. Results are shown in FIG. 29. FIG. 29 illustrates data of the subjects according to the seventh example. In FIG. 29, the horizontal axis represents the MMSE score and the vertical axis represents the number of subjects. That is, FIG. 29 illustrates the number of subjects for each MMSE score.

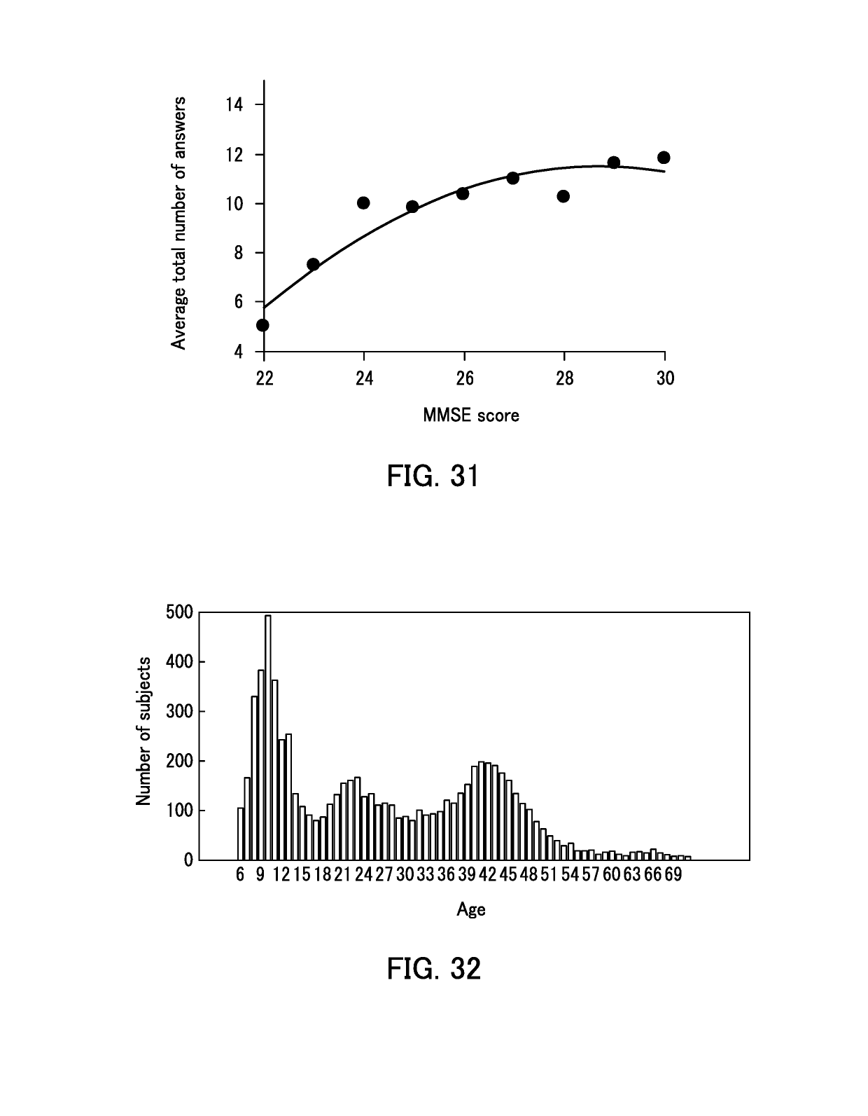

Thereafter, each subject performed a dual task of answering “calculation problems each using a one-digit number and a two-digit number” while walking in place for 45 seconds. Then, a right answer ratio and a total number of answers to the calculation problems were calculated for each subject. Also, an average value of the right answer ratio and an average value of the total number of answers were calculated for each MMSE score. Results are shown in FIGS. 30 and 31.

FIG. 30 illustrates the average value of the right answer ratio for each MMSE score. In FIG. 30, the horizontal axis represents the MMSE score and the vertical axis represents the average value of the right answer ratio. As illustrated in FIG. 30, the right answer ratio increases as the MMSE score increases. Consequently, the results show that it is highly likely that an MMSE score of a subject can be determined by measuring a right answer ratio of the subject performing the dual task.

FIG. 31 illustrates the average value of the total number of answers for each MMSE score. In FIG. 31, the horizontal axis represents the MMSE score and the vertical axis represents the average value of the total number of answers. As illustrated in FIG. 31, the total number of answers increases as the MMSE score increases. Consequently, the results show that it is highly likely that an MMSE score of a subject can be determined by measuring a total number of answers of the subject performing the dual task.

### Eighth Example

A dual task of answering “calculation problems each using a one-digit number and a two-digit number” while walking in place for 45 seconds was imposed on about 100,000 subjects. FIG. 32 illustrates data of the subjects according to the eighth example. In FIG. 32, the horizontal axis represents the age and the vertical axis represents the number of subjects. That is, FIG. 32 illustrates the number of subjects for each age.

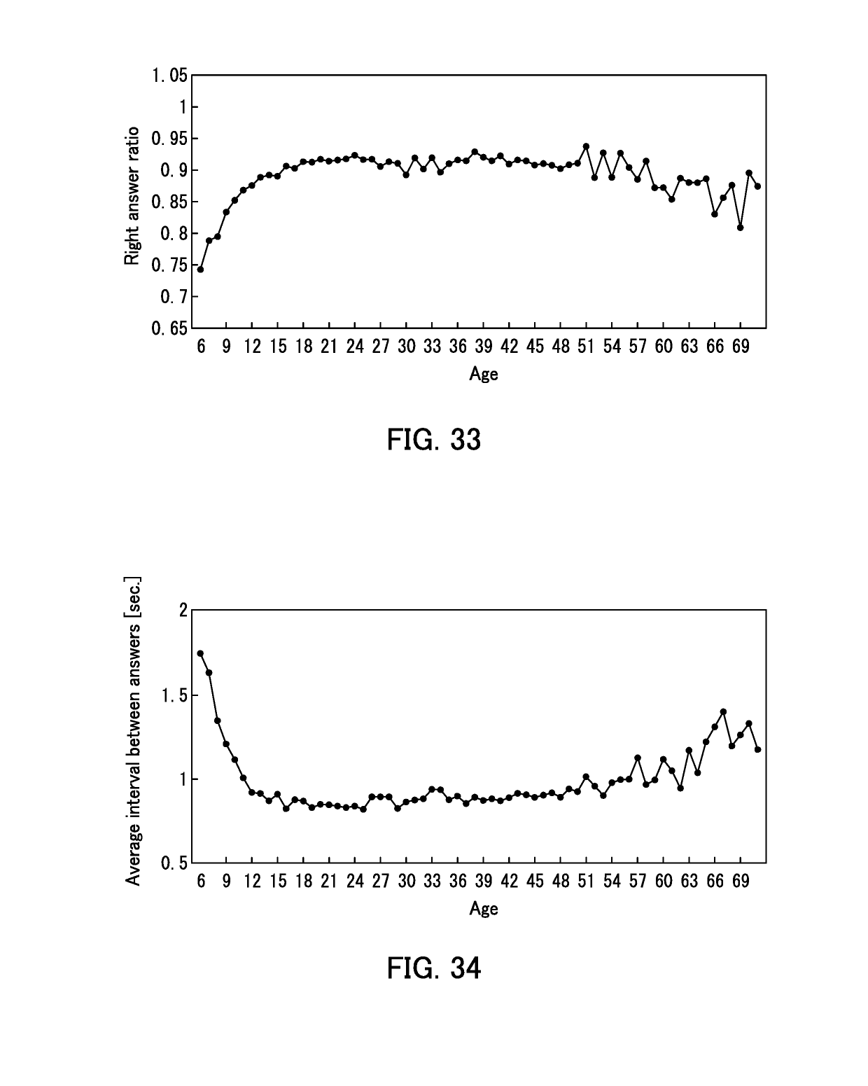

In the eighth example, a right answer ratio and an average interval between answers to the calculation problems were calculated for each subject. Also, an average value of the right answer ratio and an average value of the average interval between answers were calculated for each age. Results are shown in FIGS. 33 and 34.

FIG. 33 illustrates the average value of the right answer ratio for each age. In FIG. 33, the horizontal axis represents the age and the vertical axis represents the average value of the right answer ratio. As illustrated in FIG. 33, the right answer ratio increases as the age increases from 6 years old to 15 years old. By contrast, the right answer ratio barely changes above 15 years old. Consequently, the results show that it is highly likely that dual-task performing ability (cognitive ability, a health degree of the brain, a brain age, and the like) of a subject can be evaluated by employing data illustrated in FIG. 33 as standard value data.

FIG. 34 illustrates the average value of the average interval between answers for each age. In FIG. 34, the horizontal axis represents the age and the vertical axis represents the average value of the average interval between answers. As illustrated in FIG. 34, the average interval between answers decreases as the age increases from 6 years old to 15 years old. By contrast, the average interval between answers barely changes above 15 years old. Consequently, the results show that it is highly likely that dual-task performing ability (cognitive ability, a health degree of the brain, a brain age, and the like) of a subject can be evaluated by employing data illustrated in FIG. 34 as standard value data.

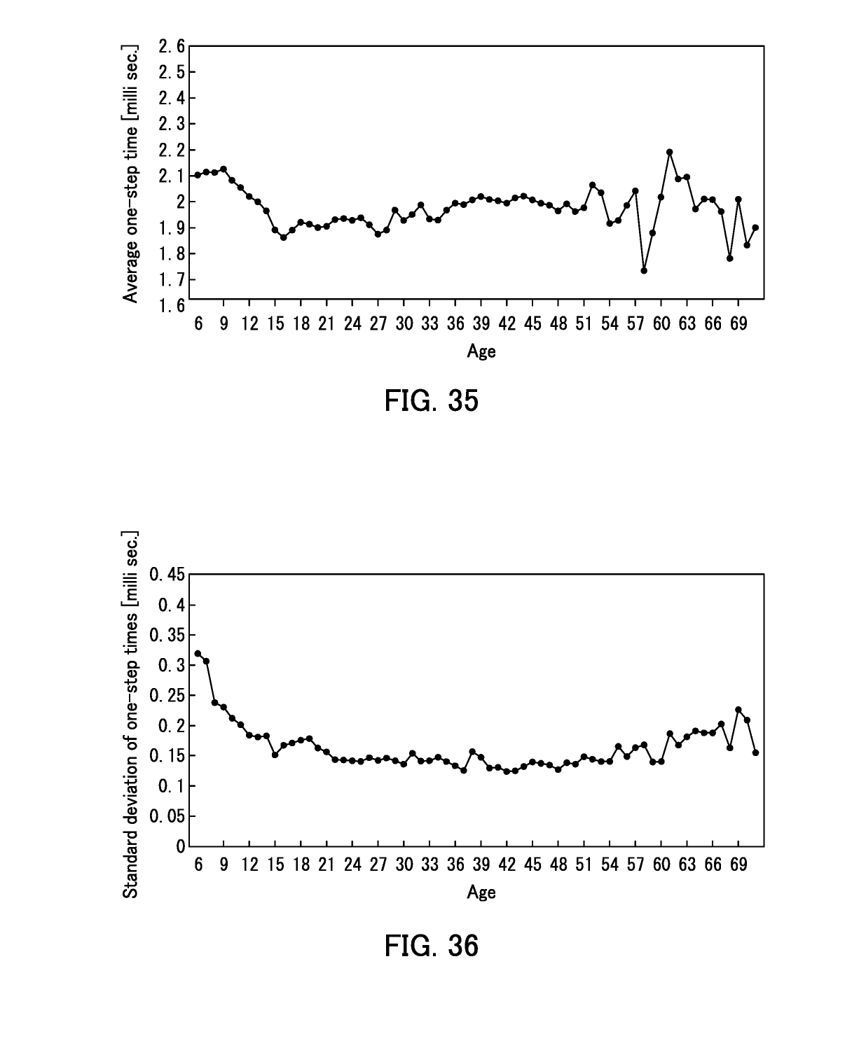

Further, in the eighth example, an average one-step time and a standard deviation of one-step times were calculated for each subject. Further, an average value of the average one-step time and an average value of the standard deviation of one-step times were calculated for each age. Results are shown in FIGS. 35 and 36.

FIG. 35 illustrates the average value of the average one-step time for each age. In FIG. 35, the horizontal axis represents the age and the vertical axis represents the average value of the average one-step time. As illustrated in FIG. 35, the average one-step time decreases as the age increases from 6 years old to 15 years old. By contrast, the average one-step time barely changes between 15 years old and 51 years old. Consequently, the results show that it is highly likely that dual-task performing ability (cognitive ability, a health degree of the brain, a brain age, and the like) of a subject can be evaluated by employing data illustrated in FIG. 35 as standard value data.

FIG. 36 illustrates the average value of the standard deviation of one-step times for each age. In FIG. 36, the horizontal axis represents the age and the vertical axis represents the average value of the standard deviation of one-step times. As illustrated in FIG. 36, the standard deviation of one-step times decreases as the age increases from 6 years old to 15 years old. By contrast, the standard deviation of one-step times barely changes above 15 years old. Consequently, the results show that it is highly likely that dual-task performing ability (cognitive ability, a health degree of the brain, a brain age, and the like) of a subject can be evaluated by employing data illustrated in FIG. 36 as standard value data.

### INDUSTRIAL APPLICABILITY

The present invention is applicable to determination of cognitive ability and a health degree of the brain, and consequently applicable to diagnosis of mental disorders such as dementia.

### REFERENCE SIGNS LIST

1a-1e Dual-task performing ability evaluation system
2 Subject
3 Information processing device
4, 4a-4c Answer detecting section
5, 5a, 5b Motion detecting section
7a, 7b Task presenting section
8 System controller

1. A dual-task performing ability evaluation method comprising:
a dual task step of detecting at least one of motion and an answer of a subject performing a dual task including a movement task that requires the subject to perform specific movement and an intelligence task that requires the subject to answer a specific problem;
an analysis step of performing analysis of the at least one of the motion and the answer that has been detected; and
an evaluation step of evaluating dual-task performing ability of the subject based on a result of the analysis.

2. The dual-task performing ability evaluation method of claim 1, wherein
in the analysis step, an answer score of the subject is calculated based on the answer detected in the dual task step, and
in the evaluation step, the dual-task performing ability of the subject is evaluated based on the answer score.

3. The dual-task performing ability evaluation method of claim 1, further comprising
a movement task step of detecting motion of the subject performing only the movement task, wherein
in the analysis step, determination is made on whether or not a state of movement of the subject is consistent between the dual task step and the movement task step with respect to a specific evaluation item, based on the motion detected in the movement task step and the motion detected in the dual task step, and
in the evaluation step, the dual-task performing ability of the subject is evaluated based on a result of the determination.

4. The dual-task performing ability evaluation method of claim 1, further comprising
a movement task step of detecting motion of the subject performing only the movement task, wherein
the dual task step includes:
a first dual task step of detecting motion of the subject performing a first dual task including the movement task and a first intelligence task that requires the subject to answer a specific first problem; and
a second dual task step of detecting motion of the subject performing a second dual task including the movement task and a second intelligence task that requires the subject to answer a specific second problem,
a difficulty level of the second intelligence task is higher than that of the first intelligence task,
in the analysis step, determination is made on whether or not evaluation results of at least two of plural evaluation items each indicating a state of movement of the subject become lower in order of the movement task step, the first dual task step, and the second dual task step, based on the motion detected in the movement task step, the motion detected in the first dual task step, and the motion detected in the second dual task step, and
in the evaluation step, the dual-task performing ability of the subject is evaluated based on a result of the determination.

5. The dual-task performing ability evaluation method of claim 1, wherein
in the analysis step, determination is made on whether or not the subject stopped the specific movement based on the motion detected in the dual task step, and
in the evaluation step, the dual-task performing ability of the subject is evaluated based on a result of the determination.

6. The dual-task performing ability evaluation method of claim 1, wherein
in the dual task step, the subject performs the dual task for a specific period of time,
in the analysis step, determination is made on whether or not the subject stopped the specific movement and finished answering the specific problem before the specific period of time elapsed, based on the motion and the answer that have been detected in the dual task step, and
in the evaluation step, the dual-task performing ability of the subject is evaluated based on a result of the determination.

7. The dual-task performing ability evaluation method of claim 1, wherein
in the analysis step, variation of intervals between answers of the subject is determined based on answers detected in the dual task step, and
in the evaluation step, the dual-task performing ability of the subject is evaluated based on a result of the determination.

8. The dual-task performing ability evaluation method of claim 1, wherein
an MMSE score or a Hasegawa's Dementia Scale is determined as the dual-task performing ability.

9. The dual-task performing ability evaluation method of claim 1, wherein
in the analysis step,
a characteristic amount of the motion detected in the dual task step or a characteristic amount of the answer detected in the dual task step is measured,
comparison is made between the measured characteristic amount and a standard value for the characteristic amount, and
in the evaluation step, the dual-task performing ability of the subject is evaluated based on a result of the comparison.

10. The dual-task performing ability evaluation method of claim 9, wherein
in the analysis step, the measured characteristic amount is compared with a standard value for an actual age of the subject.

11. A dual-task performing ability evaluation system, comprising:
an answer detecting section that detects an answer of a subject performing a dual task including a movement task that requires the subject to perform specific movement and an intelligence task that requires the subject to answer a specific problem; and
an evaluation data generating section that generates evaluation data indicating dual-task performing ability of the subject based on a result of analysis of the answer detected by the answer detecting section.

12. The dual-task performing ability evaluation system of claim 11, further comprising
an analyzing section that analyzes the answer detected by the answer detecting section.

13. The dual-task performing ability evaluation system of claim 12, wherein
the analyzing section compares a characteristic amount of the answer with a standard value for the characteristic amount.

14. The dual-task performing ability evaluation system of claim 11, further comprising
a motion detecting section that detects motion of the subject performing the dual task, wherein
the evaluation data generating section generates the evaluation data based on at least one of the result of analysis of the answer detected by the answer detecting section and a result of analysis of the motion detected by the motion detecting section.

15. The dual-task performing ability evaluation system of claim 14, further comprising
an analyzing section that analyzes at least one of the answer detected by the answer detecting section and the motion detected by the motion detecting section.

16. The dual-task performing ability evaluation system of claim 15, wherein
the analyzing section compares a characteristic amount of the answer or a characteristic amount of the motion with a standard value for the characteristic amount.

17. A dual-task performing ability evaluation system comprising:
a motion detecting section that detects motion of a subject performing a dual task including a movement task that requires the subject to perform specific movement and an intelligence task that requires the subject to answer a specific problem; and
an evaluation data generating section that generates evaluation data indicating dual-task performing ability of the subject based on a result of analysis of the motion detected by the motion detecting section.

18. The dual-task performing ability evaluation system of claim 17, further comprising
an analyzing section that analyzes the motion detected by the motion detecting section.

19. The dual-task performing ability evaluation system of claim 18, wherein
the analyzing section compares a characteristic amount of the motion with a standard value for the characteristic amount.

20. The dual-task performing ability evaluation system of claim 17, wherein
the motion detecting section further detects motion of the subject performing only a movement task that requires the subject to perform the specific movement.

21. The dual-task performing ability evaluation system of claim 17, wherein
the dual task comprises plural kinds of dual tasks, and
the motion detecting section detects motion of the subject performing each of the plural kinds of dual tasks.
